(19) United States
(12) Patent Application Publication
(10) Pub. No.: US 2019/0167233 A1
(43) Pub. Date: Jun. 6, 2019

(54) METHODS AND SYSTEMS FOR ULTRASOUND ELASTOGRAPHY

(71) Applicant: THE TRUSTEES OF COLUMBIA UNIVERSITY IN THE CITY OF NEW YORK, New York, NY (US)

(72) Inventors: Elisa KONOFAGOU, New York, NY (US); Clement PAPADACCI, Paris (FR); Ethan BUNTING, New York, NY (US); Julien GRONDIN, New York, NY (US)

(73) Assignee: THE TRUSTEES OF COLUMBIA UNIVERSITY IN THE CITY OF NEW YORK, New York, NY (US)

(21) Appl. No.: 16/206,605

(22) Filed: Nov. 30, 2018

Related U.S. Application Data
(63) Continuation of application No. PCT/US2017/035471, filed on Jun. 1, 2017.
(60) Provisional application No. 62/344,260, filed on Jun. 1, 2016.

(51) Int. Cl. A61B 8/08; A61B 8/00

(52) U.S. Cl. CPC A61B 8/485; A61B 8/483; A61B 8/486; A61B 8/466; A61B 8/0883; A61B 8/4494; A61B 8/4461; A61B 8/0825; A61B 8/085; A61B 8/445

(57) ABSTRACT

The disclosed subject matter provides methods and systems for ultrasound elastography, including the use of ultrasound to assess the mechanical properties of tissue in a three-dimensional volume. An exemplary method for ultrasound elastography includes emitting at least one non-focused wave on a target, obtaining Radio Frequency (RF) signals from the non-focused wave, beamforming 3D volumes from the RF, calculating at least two 3D displacements by comparing each volume to a reference volume, and integrating the 3D displacements to create a 3D cumulative axial strain volume.

### CROSS-REFERENCE TO RELATED APPLICATIONS

This application claims priority to and is a continuation of International Patent Application No. PCT/US2017/035471, filed Jun. 1, 2017, which claims priority to U.S. Provisional Application No. 62/344,260, filed Jun. 1, 2016, the contents of which are hereby incorporated by reference in its entirety.

### NOTICE OF GOVERNMENT SUPPORT

This invention was made with government support from the National Institutes of Health under Grant Nos. R01-EB006042 and R01-HL114358. The Government has certain rights in the invention.

### BACKGROUND

In biological tissues, an increase in stiffness can be a marker of an abnormality, and indicative of an underlying disease or condition. For example, stiffer tissue can be indicative of various tissue diseases and tumors. Thus, ultrasound elastography can be used to assess certain mechanical properties of soft tissue and can be of interest in the detection of solid tumors or other diseases resulting in a change is tissue stiffness as compared to surrounding or healthy tissue.

Techniques such as quasi-static ultrasound elastography can assess the strain distribution in soft tissues in two dimensions using a quasi-static compression. However, tumors can exhibit heterogeneous shapes and a single cross-sectional strain map can lead to an erroneous estimation of the tumor volume. Thus, a three-dimensional approach can be desirable to measure the volume with improved accuracy and to remove operator dependency. A high volume rate acquisition can also be desirable, for example to avoid signal decorrelation, to perform strain mapping in 3D with a freehand compression. Thus, there remains a need for improved techniques and systems for imaging of the elasticity in human bodies in three dimensions using simple freehand scanning.

### SUMMARY

The disclosed subject matter provides methods and systems for 3D ultrasound quasi-static elastography.

In certain embodiments, an exemplary method for ultrasound elastography includes emitting at least one non-focused wave on a target, obtaining Radio Frequency (RF) signals of the non-focused wave, beamforming 3D volumes from the RF, calculating at least two 3D displacements by comparing each 3D volume to a reference volume, and integrating the 3D displacements to create a 3D cumulative axial strain volume.

In certain embodiments, the non-focused wave can be a plane wave. The non-focused wave can be emitted at a rate of about 100 volumes/sec.

In certain embodiments, the method can include providing a compression on the target. The compression can include a natural compression, a motorized compression, a manual compression, a compression by an external source, and a compression by an ultrasound probe.

In certain embodiments, the RF can be recorded at a frequency of from about 2.5 MHz to about 10 MHz.

In certain embodiments, the beamforming can include performing a delay- and sum beamforming.

In certain embodiments, the 3D displacement comprises an axial, a lateral, and/or an elevational direction. The 3D displacement is configured to be calculated between two successive volumes.

In certain embodiments, the method can include determining a Lagrangian strain tensor, and can be utilized to detect and treat a breast cancer.

In certain embodiments, an exemplary system for ultrasound elastography, includes

a 2D matrix array probe and a signal processor. The 2D matrix array probe can include an ultrasound transducer which can emit at least one non-focused wave on a target. The signal processor can obtain at least two Radio Frequency (RF) signals from the non-focused wave, beamform 3D volumes from the RF, calculate at least two 3D displacement by comparing each 3D volume to a reference volume and integrate the 3D displacements to create a 3D cumulative axial strain volume.

In certain embodiments, the 2D matrix array probe can include a plurality of elements. The 2D matrix array can emit a non-focused wave at rate of about 100 waves per second. In certain embodiments, the ultrasound transducer can have a central frequency of about 2.5 MHz. As embodied herein, the 2D matrix probe can be configured to receive at least two Radio Frequency (RF) signals from the non-focused wave and transmit the RF signals to the signal processor.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. A method of ultrasound elastography according to an exemplary embodiment of the disclosed subject matter.

FIGS. 2A-2E. Examples setup ultrasound elastography according to exemplary embodiments of the disclosed subject matter.

FIGS. 3A-3E. Schematic illustrations of process for the elastography with plane waves framework according to exemplary embodiments of the disclosed subject matter.

FIGS. 4A-4D. Diagrams of (A) 3D B-mode volume and (B) strain volume in (C) x-z plane and (D) x-y plane.

FIGS. 5A-5D. Diagrams of (A) 3D B-mode volume, (B) 3D axial strain volume and (C-D) slices of the strain volume of the stiff inclusion embedded in the soft phantom.

FIGS. 6A-6D. Diagrams of (A) 3D B-mode volume, (B) 3D axial strain volume and (C-D) slices of the strain volume of the stiff inclusion embedded in the stiff phantom.

FIGS. 7A-7H. Diagrams of (A, E) 3D B-mode volumes, (B, F) 3D strain volumes and (C, D, G, H) slices of the strain volumes of an ex vivo canine liver before and after an HIFU ablation.

FIGS. 8A and 8B. Diagrams of strain distribution in the 3D strain volumes (A) before ablation and (B) after ablation.

FIGS. 9A-9F. Diagrams of (A) 3D B-mode volume and (B-C) slices and (D) 3D strain volume and (E-F) slices of an in vivo calf muscle of a human volunteer.

FIG. 10. A schematic illustration of electromechanical model of the human heart.

FIGS. 11A-11C. Schematic illustrations of (A) 3D simulation configuration, (B) flow chart of 3D EWI and (C) process of the 3D simulation according to exemplary embodiments of the disclosed subject matter.

FIGS. 12A-12C. Diagrams of (A) displacement estimation, (B) electromechanical strain estimation, and (C) comparison between true and estimated electrical activation times.

FIGS. 13A and 13B. Schematic diagrams of (A) 2:1 multiplexer for 3D ultrasound and (B) in vive electromechanical strain in a canine.

FIGS. 14A-14G. Flow charts of electromechanical wave imagings in accordance with an embodiment of the disclosed subject matter.

FIGS. 15A and 15B. Axial strain images obtained from benchmark mechanical finite-element simulation in the infarct-free baseline; FIGS. 15C and 15D. Axial strain images estimated from increased frame-rate 4D ultrasound simulation in the infarct-free; FIGS. 15E and 15F. Axial strain images obtained from benchmark mechanical finite-element simulation in infarcted hearts; and FIGS. 15G and 15H. Axial strain images estimated from increased frame-rate 4D ultrasound simulation in infarcted hearts, in the right (RV) and left (LV) ventricles at different time points are shown.

FIGS. 16A-16E. Electromechanical wave imagings of a canine during normal sinus rhythm at (A) 52 ms, (B) at 122 ms, (C) at 172 ms, (D) at 192 ms, and (E) at 292 ms; FIG. 16F. An electromechanical wave imaging of electromechanical activation time of the heart.

FIGS. 17A-17F. Electromechanical wave imagings of a canine during apical pacing at (A) 25 ms, (B) at 52 ms, (C) at 81 ms, (D) at 109 ms, (E) at 136 ms, (F) at 165 ms; FIG. 17G. Electromechanical wave imaging of electromechanical activation time of the heart; FIG. 17H. Electrical activation map of the epicardium obtained from electro-anatomical mapping; FIG. 17I. A picture of the heart with the pacing electrode.

FIGS. 18A-18D. Electromechanical wave imagings of a canine during left ventricular (LV) pacing (A) at 42 ms, (B) at 88 ms, (C) at 134 ms, and (D) at 180 ms; FIG. 18E. Electromechanical wave imaging of electromechanical activation time of the heart; FIG. 18F. A picture of the heart with the pacing electrode.

FIGS. 19A-19E. Electromechanical wave imagings of a human left ventricle (LV) during normal sinus rhythm (A) at 10 ms, (B) at 35 ms, (C) at 60 ms, (D) at 85 ms, and (E) at 110 ms; FIG. 19F. Electromechanical wave imaging of electromechanical activation time of the heart.

FIGS. 20A-20E and 20G-20K. In vivo electromechanical wave imagings of an infarcted canine during sinus rhythm (A-E) and during ventricular tachycardia (G-K) at different time points; FIG. 20F. Comparison of electrical and electromechanical activation times of the left ventricular; FIG. 20L. Comparison of electrical and electromechanical activation times of the ventricular tachycardia.

FIGS. 21A-21E and 21G-21K. In vivo electromechanical wave imagings of a cardiac resynchronization therapy (CRT) patient during (A-E) right ventricular and (G-K) biventricular pacing at different time points; FIG. 21F. Electromechanical activation times map of the right ventricular; FIG. 21L. Electromechanical activation times map of the biventricular pacing.

FIG. 22A. A schematic diagram of an example setup ultrasound elastography according to one exemplary embodiment of the disclosed subject matter; FIG. 22B. A schematic of radio-frequency (RF) ablation catheter on top of the heart at the same location to perform a lesion; FIG. 22C. A schematic diagram of diverging waves acquired on the left ventricle at the lesion location.

FIGS. 23A and 23B. Schematic diagrams of (A) the 3D B-mode and (B) 3D mask generation based on the 3D B-mode in accordance with an embodiment of the disclosed subject matter.

FIGS. 24A-24D and 24F-24I. Diagrams of incremental displacements and incremental strains of a portion of the left ventricle of the heart of an open-chest canine: (A, F) during mid-diastole, (B,G) during the QRS, (C,H) during early systole, and (D,I) during early diastole. FIGS. 24E and 24J. Overlaid images on the Motion-mode (M-mode), displacement and strain M-modes of the central line of the volumes with the co-registered ECG

FIGS. 25A-25G. Diagrams of cumulative displacement and cumulative strain of the left ventricle over (A, D) the diastolic phase in three dimensions, (B, C, E, and F) the middle slices of the three dimension volumes, and (G) associated B-mode.

FIGS. 26A-26G. Diagrams of cumulative displacement and cumulative strain of the left ventricle over (A, D) the systolic phase in three dimensions, (B, C, E, and F) the middle slices of the three dimension volumes, and (G) associated B-mode.

FIGS. 27A-27D and 27F-27I. Diagrams of incremental displacements and incremental strains of a portion of the left ventricle of the heart of an open-chest canine after performing radio-frequency ablation on the anterior wall of the heart: (A,F) during mid-diastole, (B,G) during the QRS, (C,H) during early systole and (D,I) during early diastole (D,I); FIGS. 27E and 27J. Overlaid images on the Motion-mode (M-mode), displacement and strain M-modes of the central line of the volumes with the co-registered ECG.

FIGS. 28A-28H. Diagrams of cumulative displacement and cumulative strain of the left ventricle after performing RF ablation on the anterior wall of the heart over (A, D) the diastolic phase in three dimensions, (B, C, E, and F) the middle slices of the three dimension volumes, (G) gross pathology, and (H) associated B-mode.

FIGS. 29A-29G. Diagrams of cumulative displacement and cumulative strain of the left ventricle over (A, D) the systolic phase after performing RF ablation on the anterior wall of the heart in three dimensions, (B, C, E, and F) the middle slices of the three dimension volumes, and (G) associated B-mode.

FIGS. 30A-30D. Graphs of cumulative displacement average with associated standard deviation and cumulative strain average with associated standard deviation in (A, C) the anterior and (B, D) posterior walls over the systolic and diastolic phases before and after RF ablation.

FIGS. 31A-31D. Graphs of (A) incremental displacements, (B) cumulative displacements, (C) incremental strains, and (D) cumulative strains at one point of the anterior wall before and after ablation.

### DETAILED DESCRIPTION

The disclosed subject matter provides methods and systems for ultrasound elastography, including the use of ultrasound to assess the mechanical properties of tissue in a three-dimensional volume. In certain embodiments, the disclosed methods and systems provide three-dimensional (3D) ultrasound elastography using non-focused waves.

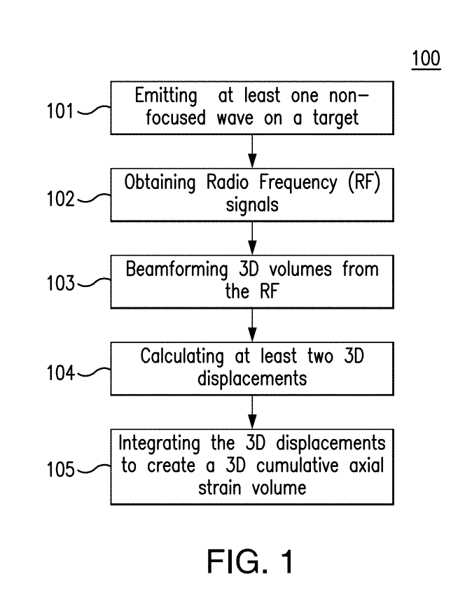

For example and not limitation, FIG. 1 provides a schematic illustration of an exemplary method for 3D ultrasound elastography. In certain embodiments, a method 100 includes emitting at least one non-focused wave to a target 101. In certain embodiments, the target can include any biological tissue, as known in the art. For example, the non-focused wave can be emitted to soft tissues such as muscles, tendons, ligaments, fascia, connective tissue, fat, heart, skin, liver, breast, prostate, thyroid, etc.

As embodied herein, the non-focused waves can be in various forms, including plane waves, circular waves, and/or spherical waves. In certain embodiments, the non-focused waves can be emitted by an ultrasound transducer. For example, the ultrasound transducer can be configured to emit ultrasound waves at a high volume rate, for example and as embodied herein, at a rate of from about 50 volumes/s to about 1000 volumes/s, or from about 100 volumes/s to about 500 volumes/s. In particular embodiments, the rate of ultrasound wave emission can be about 100 volumes/s or about 500 volumes/s.

As embodied herein, the ultrasound transducer can emit waves for a certain period of time. As such, a known number of wave volumes can be emitted over that certain period of time based on the volume rate. By way of example, and not limitation, an ultrasound transducer operating at a volume rate of 100 volumes/s can be configured to emit waves for 1 second or more such that at least 100 plane waves are emitted. As embodied herein, for example and not limitation, the method can include emitting at least 20 plane waves, at least 50 plane waves, at least 100 plane waves, at least 250 plane waves, or at least 500 plane waves.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and or up to 1% of a given value.

With further reference to FIG. 1, the method 100 can further include recording a Radio Frequency (RF) of the non-focused wave 102. In certain embodiments, the RF can be a backscattered echo from an ultrasound wave, e.g., a plane wave. For example, backscattered echoes from each emitted ultrasound wave can be recorded at a frequency of from about 2.5 MHz to about 10 MHz, e.g., at about 10 MHz. As embodied herein, the recorded RF can optionally be stored in memory.

As embodied herein, the method 100 can further include beamforming 3D volumes from the RF 103. In certain embodiments, the beamforming can be performed using a delay-and-sum algorithm, as is known in the art. By way of example, and not limitation, 3D B-mode volumes can be beamformed from the RF.

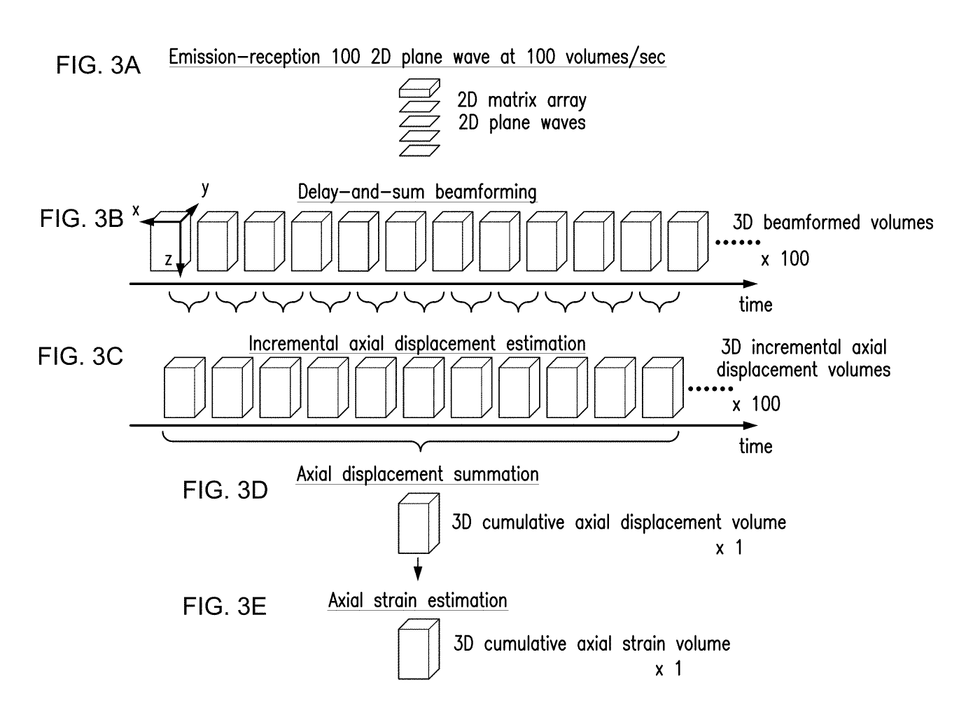

As shown for purposes of example in FIG. 3, multiple 3D volumes can be generated from two or more RFs. Each 3D volume can correspond to one or more RFs from a single ultrasound wave. As such, in embodiments where multiple plane waves are emitted, a 3D volume can be beamformed to correspond to each plane wave.

The method 100 can further include calculating at least one 3D displacement compared to a reference volume 103. For example, the 3D displacements, such as displacement in an axial, a lateral, and/or an elevational direction, can be determined as between two successive 3D volumes. Thus, a first 3D volume beamformed from a first ultrasound wave can form the reference volume for a second 3D volume beamformed from a second ultrasound wave. Where the waves are emitted from the same position at different times, the 3D displacement can reflect changes in the tissue geometry over time. Alternatively, where the waves are emitted from different positions, the 3D displacement can reflect changes in the tissue geometry in space. As embodied herein, the 3D displacement can be estimated by normalized 1D and/or 3D cross-correlation and/or re-correlation, as known in the art.

The method 100 can further include integrating the 3D displacements to create a 3D cumulative axial strain volume 104. For example, the 3D incremental axial displacements can be integrated over time to obtain 3D cumulative axial displacements, as shown in FIG. 3. Then, the 3D cumulative displacements can be filtered using a 3D median filter. The 3D cumulative axial strain distributions can be estimated from the 3D cumulative displacements by applying a least-squares estimator. The final 3D axial strain volume can be displayed graphically. Optionally, more than one 3D median filter can be applied on the 3D cumulative displacement volume or/and 3D strain volume. Lagrangian axial, lateral, and elevational strain volumes can be computed from the cumulative displacement volumes. In certain embodiments, a coordinate transformation can then be applied to retrieve circumferential, radial, and longitudinal strain volumes for in vivo applications such as the heart. In some embodiments, the presently disclosed techniques and systems can further provide the strain distribution in the elevational direction. As embodied herein, anisotropy and/or elasticity values can be determined from the elevational strain.

In certain embodiments, the method can include emitting ultrasound waves to create 3D cumulative axial strain volumes at different positions in the target tissue. For example, the method can be repeated at two or more positions to obtain multiple 3D cumulative strain volumes. The multiple 3D cumulative strain volumes can be assembled and/or integrated to obtain a single graphical representation of the 3D cumulative strain.

The method 100 can optionally further include providing a compression on the target prior to the emitting. The compression can include a natural compression, a motorized compression, a manual compression, a compression by an external source, and a compression by an ultrasound probe. For example, compression can be provided manually using freehand techniques, e.g., massage, with or without the use of a tool to apply compression. Additionally, or alternatively, uniaxial compression can be applied using a linear motor. In certain embodiments, up to a 3% compression at a motor speed of 3% compression per second can be applied, although a person of skill in the art will appreciate that the magnitude and speed of compression can be adjusted to the desired application. Similarly, a stepper motor can be attached to the ultrasound probe and the compression can be applied by the ultrasound probe mechanically through the motor. Alternatively, compression can be applied by manually pressing the ultrasound probe. For non-moving organs, an external compressor can be applied on the target to induce deformation. For moving organs such as the heart, natural contraction can be used as the deformation. In certain embodiments, the compression can be provided prior to or/and during emission of the non-focused wave. The compression can be applied to the target continuously, intermittently, and/or incrementally.

In certain embodiments, the method can further include beamforming the RF signals to calculate inter-volumes displacements and strains. The displacements and strains over time can be analyzed to identify zero-crossings of the strain curve, i.e., locations and time points at which the displacements switch direction. Such zero-crossings can be indicative of electromechanical activation. In some embodiments, the disclosed method can further include interpolating the zero-crossings into the series of isochrone maps to show the electromechanical activation over time. For example, a delay-and-sum beamforming, with a predetermined axial sampling (e.g., 32.1 μm), a predetermined sector angle (e.g., 90°), and a density of one line per degree in both lateral and elevational directions can be performed. 1-D (axial) normalized cross-correlation (e.g., 5 mm window, 70% overlap) of beamformed RF signals can be used to estimate inter-volumes displacements. The axial inter-volume strains can be obtained using a least-squares estimator. The axial inter-volume strains can be mapped to volume of voxels (e.g., 256×256×256 voxels).

In non-limiting embodiments, the isochrone map can be generated by interpolation of the zero-crossings to show electromechanical activation over time. Onset of longitudinal shortening, resulting from electrical activation, can be determined by the time of first positive-to-negative zero-crossing of the axial strain curves after a reference time point and be defined as the electromechanical activation time. For example, a random selection of 1,000,000 voxels from the reconstructed myocardial volume can be performed and for each voxel selected, the time of first positive-to-negative zero-crossing after the reference time point can be automatically computed. During normal sinus rhythm, the reference time point can be set to the onset of the P-wave for voxels located in the atria and to the onset of the QRS complex for voxels located in the ventricles. During ventricular pacing, the reference time point can be set to the time of pacing. The electromechanical activation times can be linearly interpolated in the myocardial volume to obtain the electromechanical activation map or isochrone. The in vivo electromechanical map can be replicated for two consecutive cardiac cycles.

In non-limiting embodiments, for in vivo imaging, segmentation can be performed from focused 2D ultrasound images acquired after the diverging wave imaging sequence. For example, focused 2D image acquisitions can be performed using the 2D array aperture and by steering the focused beam in the lateral direction over a 90° sector angle with a beam density of one line per degree. The epicardial and endocardial contours as well as the atrial and ventricular septum can be segmented from the 2D ultrasound image. In some embodiments, the segmentation in the elevational direction can be performed by assuming central symmetry of the left atria and ventricle and of the epicardial and endocardial contours at each depth. By orienting the transducer in the apex-to-base direction, the direction of propagation of the ultrasound beam can be aligned with the longitudinal direction of the heart.

In certain aspects, the present disclosure further provides systems for ultrasound elastography. For example, the presently disclosed systems can provide a device for ultrasound elastography including a 2D matrix array probe and a signal processor. The 2D matrix array probe can be coupled to an ultrasound transducer to emit at least one non-focused wave on a target. As used herein, the word “coupled” means directly connected together or connected through one or more intervening elements. The connection can be a physical connection or an operable connection, e.g., such that a signal or wave can be transmitted between the elements. For example, the 2D matrix array probe can be connected to emission and receive channels of the ultrasound transducer to emit and receive ultrasound waves and/or Radio Frequency (RF) signals. The 2D matrix probe can receive at least two RF signals from the non-focused wave and transfer the RF signals to a signal processor. The 2D matrix array probe can be fully programmable and can include a plurality of elements. In certain embodiments, the 2D matrix array probe can include at least about 100 elements, at least about 200 elements, at least about 250 elements, at least about 500 elements, or at least about 1000 elements. For example, and not limitation, the probe can include an array of elements, which can include about 256 elements (e.g., 16×16) or about 1024 elements (e.g., 32×32) to create a programmable ultrasound system with about 256 or about 1024 channels.

In certain embodiments, the system can include a multiplexer (e.g., a 2:1 multiplexer) in transmit and/or receive channels. For the purpose of example and not limitation, the system can include a combination of two or more ultrasound scanners and the multiplexer can be used to interface the channels of the ultrasound scanners and elements of the ultrasound transducer. For example, in an ultrasound system that has two ultrasound scanners containing 256 channels each, a 2:1 multiplexer can be used to interface the 1024 element probe with a 512 total channels on the ultrasound scanners. In such embodiments, the 2:1 multiplexer can switch between 2 positions to interface the elements of the transducer with channels on the scanner, e.g., elements 1 through 512 or 513 through 1024.

In certain embodiments, the signal processor can record a RF of the non-focused wave, beamform a 3D volume from the RF, calculate at least one 3D displacement, and/or integrate the 3D displacements to create a 3D cumulative axial strain volume. The ultrasound system can have a central frequency of about 2.5 MHz. The signal processor can include an ultrasound scanner, as described above.

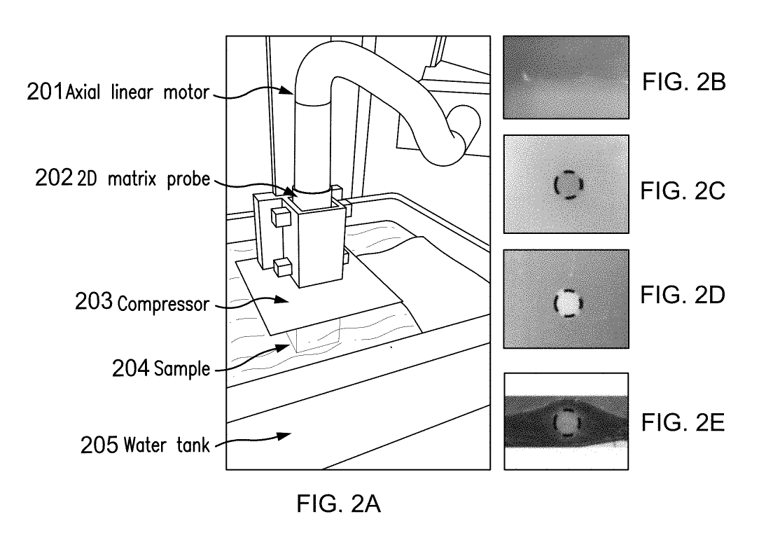

For the purpose of illustration, FIG. 2 provides an example schematic of the operation of a system according to the disclosed techniques. As shown in FIG. 2A, a 2D matrix array probe 202 can be mounted on an axial linear motor 201. A compressor 203 fitted with the footprint of the probe can be attached to the bottom of the probe. The target samples 204 can be positioned underneath. The target can optionally be immersed in a water tank 205 to conduct waves from the ultrasound probe.

The presently disclosed techniques and systems can be used to detect abnormalities in tissue. For example, these techniques can be used to detect various types of cancer, including breast cancer. Alternatively, or additionally, the techniques can be used to monitor myocardial function, for example for ischemia and infract detections and three-dimensional myocardial ablation lesion monitoring. The presently disclosed systems and techniques can also be used to monitor radiofrequency ablation therapy for cardiac arrhythmia and high intensity focused ultrasound (HIFU) therapy for various cancers. By way of example, and not limitation, the presently disclosed techniques and systems can be utilized in the diagnosis of various diseases and disorders, including, but not limited to, atrial fibrillation, atrial flutter, ventricular tachycardia, and heart failure. In certain embodiments, the presently disclosed techniques and systems can be utilized for cardiac imaging, Doppler imaging, electromechanical wave imaging, and/or shear wave imaging.

In certain embodiments, the presently disclosed techniques and systems can decrease intra- and inter-observer variability that can occur when a 2D plane is imaged. For example, the presently disclosed techniques and systems can alleviate certain signal de-correlation between the first and the last 2D image acquired in the stack of 2D images contained in the 3D volume; or between the reconstructed 3D volumes before compression and after compression due to the short time needed to acquire 3D volumes. This decorrelation can be due to hand motion (in case of freehand compression), normal physiologic motions induced by cardiac or respiratory activities, or to a too high strain amplitude between the two volumes. Signal decorrelation can affect a signal-to-noise ratio (SNR) of the strain images. The presently disclosed techniques can acquire and process backscattered ultrasound emissions to estimate 3D axial strain volumes and prepare 3D images of axial strain in real time.

In certain embodiments, the presently disclosed technique and system can provide noninvasive 4D mapping of the cardiac electromechanical activity in a single heartbeat. The disclosed technique can capture the activation wave throughout all four chambers in vivo. For example, electromechanical activation maps can be presented in a normal and infarcted cardiac model in silico and in canine heart during pacing and re-entrant ventricular tachycardia in vivo. Furthermore, noninvasive 4D electromechanical activation mapping in a healthy volunteer and a heart failure patient can be performed. In some embodiments, the noninvasive 4D mapping technique can be implemented on a clinical ultrasound system for direct visualization of electromechanical activation of the heart at the early onset of heart dysfunction, for timely and effective treatment.

In certain embodiments, the system can include a 2D array including a plurality of transducer elements. For example, the ultrasound system can be a 2:1 multiplexer. The ultrasound system can image the heart by performing a diverging imaging sequence and/or a focused wave imaging sequence. In some embodiments, the methods can include acquiring up to 4 apical views. The ultrasound can be configured with an echographic system to acquire and record wave images. For example, a custom-made 2D array of 32×32 ultrasound elements at 3 MHz center frequency and with a pitch of 0.3 mm can be used for 4D ultrasound imaging. The 2D array, which can have a total of 1024 transducer element, can be connected to at least one ultrasound system, each of which having 256 channels. In order to use all the elements of the 2D array, a 2:1 multiplexer can be used for switching from one half of the array to the other half. In some embodiments, diverging and/or focused wave imaging sequences can be used to image the heart. The ultrasound probe can be positioned to acquire apical views of the heart and fixed to a support to maintain the same echocardiographic view for diverging and focused acquisitions. For example, for diverging wave imaging, an ultrasound source 203 can be placed 4.8 mm behind the surface of the transducer to allow for increased volume-rate imaging. The first and the second halves of the 2D array aperture can be used alternatively for each transmit-receive event at a pulse repetition frequency of 1000 Hz. Two consecutive transmit-receive events using each half of the aperture were can be used to reconstruct an entire image, yielding an imaging rate of 500 volumes per second. The ultrasound channel data can be acquired during 2 s.

In certain embodiments, the system can generate a heart model for an electromechanical simulation and an ultrasound simulation based on the series of image frames and radio frequency (RF) signals corresponding to the location in the heart. The heart model can illustrate an activation and a propagation of a cardiac action potential.

In certain embodiments, the disclosed subject matter can provide an electromechanical heart model including an electrophysiological model and a mechanical model. The electrophysiological model can illustrate the activation and propagation of cardiac action potentials by using a reaction-diffusion partial differential. The mechanical model can illustrate the heart deformation by the balance-of-force equation in which the active contraction force of myofilaments drives the cyclic heart motion. The electrophysiological and mechanical models can be coupled at the cellular level: the membrane ionic kinetics regulates the intracellular calcium transient, which dictates myofilament contraction. The electromechanical simulation can be conducted based on a MRI-image-based finite element human heart model with rule-based fiber structures, with the myocardial tissue regarded as an orthotropic, hyperelastic, and nearly-incompressible material. The simulation result can show the deformation of a human heart without and with infarct, both during sinus rhythm. In non-limiting embodiments, the inter-volume axial (apex-to-base direction) displacements obtained from the mechanical finite-element simulation can be sampled, with of 1.8 ms between samples.

In some embodiments, an ultrasound simulation program can be used to generate the ultrasound radio-frequency channel signals with a simulated transducer and a distribution of scatterers. The scatterers can be uniformly distributed at random positions in the ventricles, which geometry is obtained from the mechanical finite-element simulation at each sample. In order to perform 4D ultrasound imaging, a 2D array of 32×32 transducer elements, with an inter-element spacing (or pitch) of 0.3 mm and a center frequency of 3 MHz can be simulated. Increased volume-rate imaging can be achieved by using parallel beamforming with diverging wave imaging, for which the entire volume can be reconstructed from a single transmitted beam. For example, the emission of a spherical wavefront with a transmit angle aperture of 90° can be achieved by placing a virtual source 4.8 mm behind the surface of the transducer. In certain embodiments, a delay-and-sum algorithm can be used to reconstruct the image with an axial sampling of 32.1 μm, a sector angle of 90° and a density of one line per degree in both lateral and elevational directions. The scatterers can be displaced according to the displacement field obtained from the mechanical finite-element simulation at each time sample. The ventricles were imaged at each sample (1.8 ms) during an entire heartbeat, which entails an imaging rate of 556 volumes per second.

In certain embodiments, the disclosed subject matter provides a technique to noninvasively map myocardial activation of the full heart in a single heartbeat throughout all four chambers in 4D (3D over time). This technique can image cardiac deformation resulting from electrical activation with increased frame-rate (500-2000 frames per second) ultrasound to derive the electromechanical activation of the heart. The propagation of myocardial shortening can be described as an electromechanical wave, as it consists of mechanical deformations directly resulting from their local, respective electrical activations. An activation map obtained from this technique can be used as a surrogate for cardiac electrical mapping. Furthermore, this technique can allow to link the electrical and mechanical functions of the heart. Such linking can have a clinical value. In some embodiments, the disclosed technique provides a 4D increased frame-rate ultrasound technique to image the propagation of the electromechanical wave and derive the activation map throughout all four chambers simultaneously.

In certain embodiments, the disclosed subject matter can provide systems and methods for 3D myocardial elastography at high volume rates using diverging wave transmits to evaluate the local axial strain distribution in three dimensions in three open-chest canines before and after radio-frequency ablation. For example, acquisitions can be performed with a 2D matrix array fully programmable used to emit about 2000 diverging waves at about 2000 volumes/s. Incremental displacements and strains during the QRS complex can be visualized along with the different phases of the cardiac cycle in entire volumes. In some embodiments, cumulative displacement and strain volumes can depict a contrast between non-ablated and ablated myocardium at the lesion location, mapping the tissue coagulation.

### EXAMPLES

The presently disclosed subject matter will be better understood by reference to the following Examples. These Examples are provided as merely illustrative of the disclosed methods and systems, and should not be considered as a limitation in any way.

### Example 1: 3D Quasi-Static Ultrasound Elastography with Plane Waves

This Example describes one exemplary method of 3D quasi-static ultrasound elastography methods with plane waves using parallel receive beamforming that estimates axial strain distribution in vivo in entire volumes at a high volume rate, e.g., 100 volumes/sec or 500 volumes/sec.

Methods

A fully programmable ultrasound system with 256 fully programmable channels in emission and receive (Vantage, Verasonics, Kirkland, USA) was used to control an ultrasonic 2D matrix array probe of 256 elements (16-by-16 elements), with a 0.95 mm² pitch, a central frequency of 2.5 MHz, and a bandwith of 50% (Sonic Concepts, Bothell, USA). The volume delay-and-sum beamforming and the axial strain distribution calculations were performed on a Tesla K40 GPU (Nvidia, Santa Clara, USA). 3D rendering was computed with Amira software (Visualization Sciences Group, Burlington, USA).

A. Ex Vivo Example

The 2D matrix array probe was mounted on a linear motor 201 (FIG. 2A). A square-plate compressor 203 (10 cm-by-10 cm) was designed to fit the foot-print of the probe and to compress the samples with uniaxial compression. The samples were immersed inside a water tank 205 with temperature maintained between 10° C. and 15° C. A 2D real-time focused B-mode image was used to position the probe on the samples and a low axial pre-compression was applied to the samples before starting (˜1%).

The samples were continuously compressed using a linear motor 201 up to a 3% compression at a motor speed of 3% compression per second. Simultaneously, 100 plane waves were emitted from the 2D-matrix array probe 202 at a rate of 100 plane waves per second in order to reconstruct 100 volumes for a total compression of 3% (FIGS. 3A and 3B); the radio-frequency (RF) backscattered echoes from each plane wave were recorded at a frequency of 10 MHz and stored in memory.

B. Two-Layer Phantom

The two-layer gelatin phantom used is shown in FIG. 2B. The first layer was made from 3% of gelatin (Bloom-275) resulting to a 4.2-kPa stiffness. The second layer was made from 12% of gelatin resulting to a 75.3-kPa stiffness. In both layers, corn stash was added to improve the backscattering properties of the gels; 1.2% in the first layer and 0.3% in the second layer to be able to see the difference on the 3D B-mode volume.

C. Stiff Inclusion

A stiff gelatin inclusion (12% gelatin) with a 140 mm diameter with 0.3% corn stash; was embedded in a softer gelatin phantom (3% gelatin) with dimensions 60 mm×40 mm×40 mm with 1.2% corn stash (FIG. 2C).

D. Soft Inclusion

A soft gelatin inclusion (3% gelatin) with a 140 mm diameter with 1.2% corn stash; was embedded in a stiffer gelatin phantom (12% gelatin) with dimensions 60 mm×40 mm×40 mm with 1.2% corn stash (FIG. 2D).

E. Ex Vivo Canine Liver

The feasibility of 3D quasi-static elastography was then demonstrated in an ex vivo canine liver before and after a High Intensity Focused Ultrasound (HIFU) ablation (FIG. 2E). 3D quasi-static elastography was first performed on the liver without ablation. The real-time focused B-mode image of the 2D-matrix array probe was used to manually position a needle in the middle of the plane-of-view. Then, the HIFU setup was positioned at the same location using the needle as a landmark. After HIFU ablation, the needle was repositioned at the same location enabling the 2D matrix array probe to return at its previous location. 30 minutes post-ablation, the 3D quasi-static elastography was performed on the ablation lesion location. The HIFU ablation was performed using a 93-element HIFU array (H-178, Sonic Concept Inc. Bothell Wash., USA) generating an amplitude-modulated signal (f_{carrier}=4.5 MHz and f_{AM}=25 Hz) with an acoustic power measured in water of 5.04 W. The liver sample was sonicated for 120 s, which has been shown to generate reproducible lesion.

F. In Vivo Example

The feasibility of the 3D quasi-static elastography was then demonstrated in vivo in the calf muscle of a human volunteer. The calf was set resting on a chair while the 2D matrix array probe was hand-held allowing freehand scanning.

The calf muscle was continuously and smoothly compressed using the same square compressor used previously. Similar to the ex vivo example, 100 2D-plane waves were emitted from the 2D-matrix array probe at a rate of 100 plane waves per second in order to reconstruct 100 volumes for the total freehand compression. The RF backscattered echoes from each plane wave were recorded at a frequency of 10 MHz and stored in memory.

Image Formation and 3D Strain Calculation

The 3D quasi-static elastography framework is depicted in FIG. 3. From RF data, a classical three-dimensional delay-and-sum algorithm was used to beamform a 3D volume from each plane wave acquisition, resulting in a total of 100 volumes (FIG. 3B). The lateral sizes (x and y directions) of the volumes were set to 15.2 mm corresponding to the aperture of the matrix probe whereas the reconstructed depth was set to 30 mm-60 mm depending on the application. The lateral sampling was set to 237.5 μm in x and y directions (FIG. 3B). The axial sampling was set to 61.6 μm corresponding to a λ/10 beamforming (where A is the ultrasonic wavelength). From the RF beamformed signals, B-mode volumes were displayed using a Hilbert transform and display with the Amira software. The 3D incremental axial displacements between two successive volumes were estimated by normalized 1D cross-correlation with a window size of 6.16 mm (corresponding to a 10λ window size as indicated in and a 95% overlap) (FIG. 3C). The 3D incremental axial displacements were then integrated over time to obtain 3D cumulative axial displacements (FIG. 3D). The 3D cumulative displacements were then filtered using a 3D median filter with a pixel kernel of 3 pixels-by-3 pixels-by-3 pixels (195 μm-by-712 μm-by-712 μm). 3D cumulative axial strain distributions were computed from 3D cumulative displacements by applying a least-squares estimator with a relatively small kernel of 646 μm (FIG. 3E). A second 3D median filter with a pixel kernel of 3 pixels-by-3 pixels-by-3 pixels (195 μm-by-712 μm-by-712 μm) was then applied on the strain volume. The final 3D axial strain volume was then displayed using Amira software.

Two slices in the x-z and y-z plane of the 3D axial strain volume were displayed for the case of the inclusion phantoms and the liver. A region of interest (ROI) was arbitrarily applied at the strain boundary of the two layers phantom, around the inclusions and the ablation lesion. The absolute strain values were averaged inside (Sᵢₙ) and outside (Sₒᵤₜ) the ROI with associated standard deviations and an observed strain contrast (C) was calculated according to Formula 1, below:

$$C = 20 {log}_{10} ( \frac{{S}_{out}}{{S}_{in}} ) \qquad ( 1 )$$

In the case of ex vivo liver ablation a histogram presenting the strain distribution in the volume has been calculated before and after ablation to highlight the strain increase due to ablation stiffening.

Results

Two-Layer Phantom

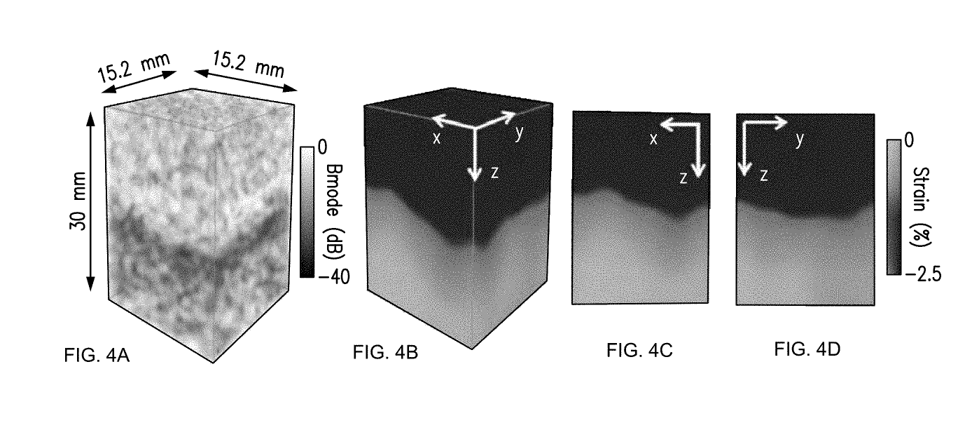

3D quasi-static elastography was first applied to a two-layer gelatin phantom. The layer at the top was made about eighteenth times softer and more echogenic than the layer at the bottom. The last 3D B-mode volume is shown in of FIG. 4A. The difference in echogeneicity is clearly visible and the two layers are distinguishable. The 3D cumulative axial strain volume following a 3% compression was formed, as described in the methods. 3D cumulative axial strain volume is shown in FIG. 4B with the associated slices in x-z plane (FIG. 4C) and in x-y plane (FIG. 4, panel D). The softer layer exhibited in average a higher absolute strain (Sᵢₙ=2.2%±0.1%) than the stiffer layer (Sₒᵤₜ=0.4%±0.2%), as it was expected. The observed strain contrast was calculated and gave a value of C=14.8 dB. The boundary between the two layers on the 3D strain volume was in agreement with the boundary found on the 3D B-mode volume.

Stiff Inclusion

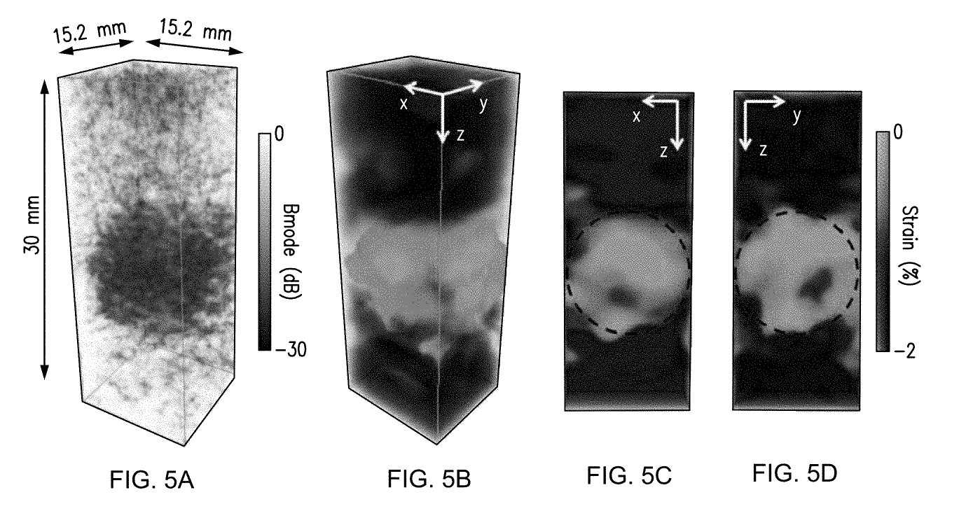

3D quasi-static elastography was then applied to a stiff gelatin inclusion embedded in an eighteenth times softer gelatin phantom. The results are displayed in FIG. 5. The inclusion was clearly identifiable on the 3D B-mode volume as it was made with a smaller concentration of scatterer (FIG. 5A). The 14 mm stiff inclusion was also identifiable on the 3D axial strain volume due to the lower absolute strain estimated at the inclusion location (Sᵢₙ=0.3%±0.4%) compared to the surrounding (Sₒᵤₜ=1.5%±0.4%) (i.e., FIGS. 5B, 5C and 5D). FIGS. 5C and 5D show the cross-section of the 3D axial strain volume in the x-z plane and the y-z plane respectively with an arbitrary circle of a 14 mm diameter depicting the ROI where the absolute strain estimates were calculated. The observed contrast (Eq. 1) C=13.9 dB, was lower than the two-layer phantom case. This can be explained by the geometry of the inclusion. Indeed, a lower contrast transfer efficiency, which represents the ratio of elasticity contrast measured from strain estimation to the true contrast, was expected in the case of a spherical inclusion. In addition, some of the predicted shadowing artifacts can be observed outside the inclusions on FIGS. 5C and 5D.

Soft Inclusion

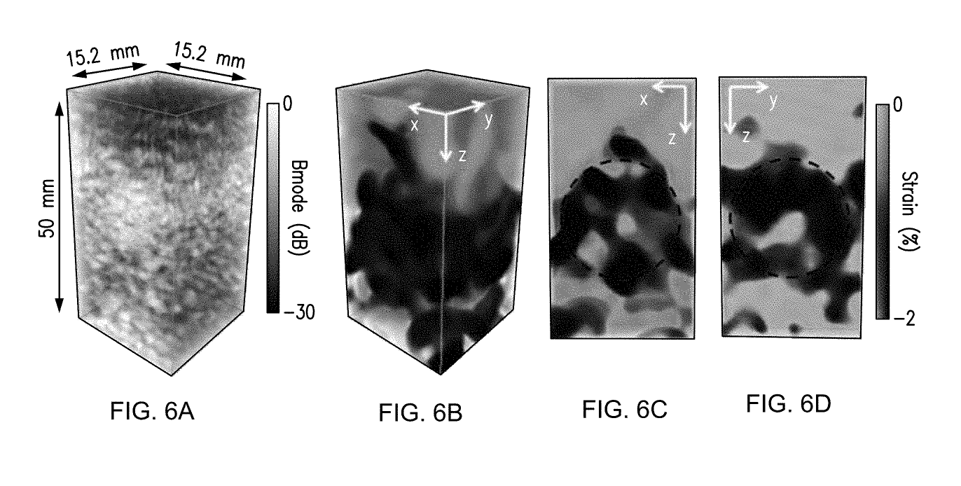

The method was then applied to a soft gelatin inclusion embedded in an eighteenth times stiffer gelatin phantom. The inclusion could not be seen on the 3D B-mode volume (FIG. 6A) due to the identical concentration of scatterer inside and outside the inclusion. However, the inclusion could be detected on the 3D axial strain volume (FIG. 6B). The higher absolute strain (Sᵢₙ=1.4%±0.8%) indicates that the inclusion is softer than the surroundings (Sₒᵤₜ=0.6%±0.7%). FIGS. 6C and 6D show the cross section of the 3D strain volume in x-z plane and y-z plane respectively with an arbitrary circle of a 14 mm diameter depicting the ROI where the absolute strain values were calculated. The observed contrast was calculated and found to be equal to: C=−7.4 dB. The minus sign indicates that this is the contrast for a soft inclusion (Eq. 1), however the absolute contrast found was significantly lower than the one for the stiff inclusion. This was predicted as the elastic modulus contrast of soft inclusion fundamentally, leads to a lower contrast transfer efficiency. The homogeneity artifacts inside the inclusions could also be explained by the low contrast transfer efficiency.

Ex Vivo Liver

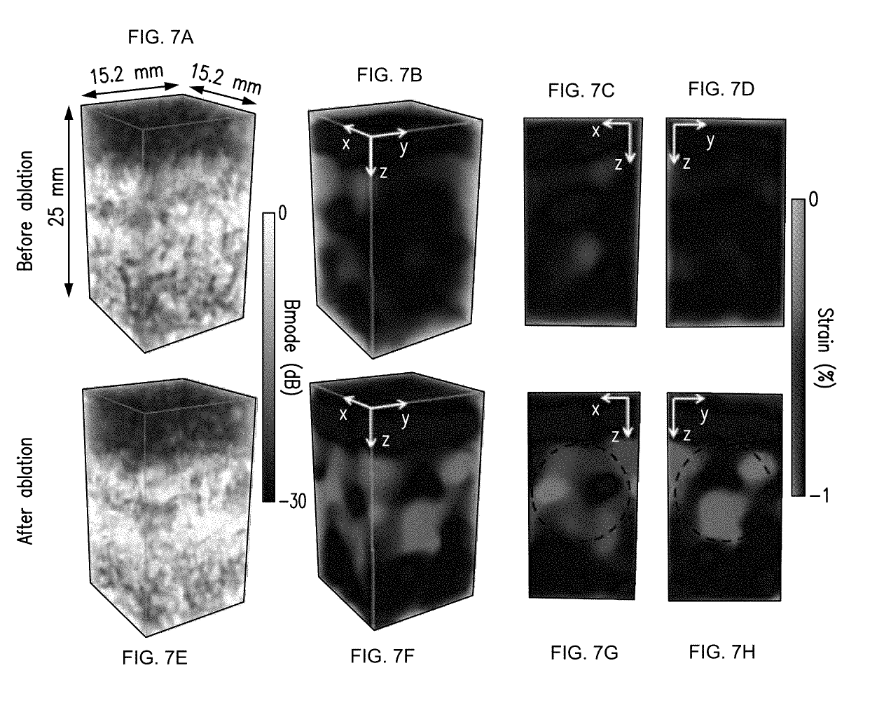

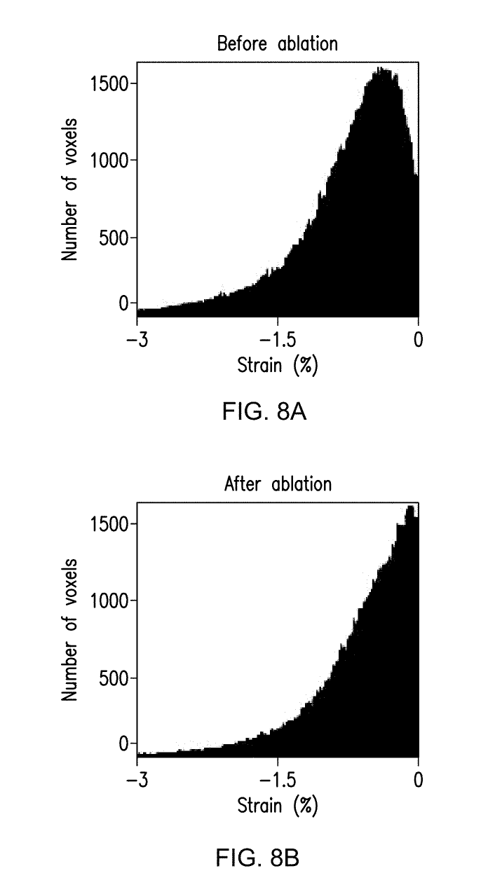

3D quasi-static elastography was obtained to an ex vivo canine liver before and after a HIFU ablation. The results are shown in FIG. 7 and FIG. 8. Before ablation, the 3D B-mode volume exhibited a heterogeneous echogenicity (FIG. 7A). This heterogeneity is also noticeable on the 3D axial strain volume (FIG. 7B) and 2D strain images (FIGS. 7C and 7D), revealing the structural complexity of the liver. After ablation, the 3D B-mode volume is more echogenic at the ablation location. On the 3D axial strain volume (FIG. 7F), one can notice a decrease on the absolute strain at the HIFU ablation location as expected. Indeed, the effect of HIFU ablation in biological tissue is an increase in stiffness. According to the average strain inside the ablation (Sᵢₙ=0.25%/1.1%) and outside the ablation (Sₒᵤₜ=0.8%/1.3%) the observed contrast C=10.1 dB was calculated and was found comparable to the stiff inclusion. However, the homogeneity artifacts are higher in this case as it is showed by the higher standard deviation from the average strain calculation. These artifacts could be due to the shadowing artifacts of a spherical inclusion.

In Vivo Calf of a Human Volunteer

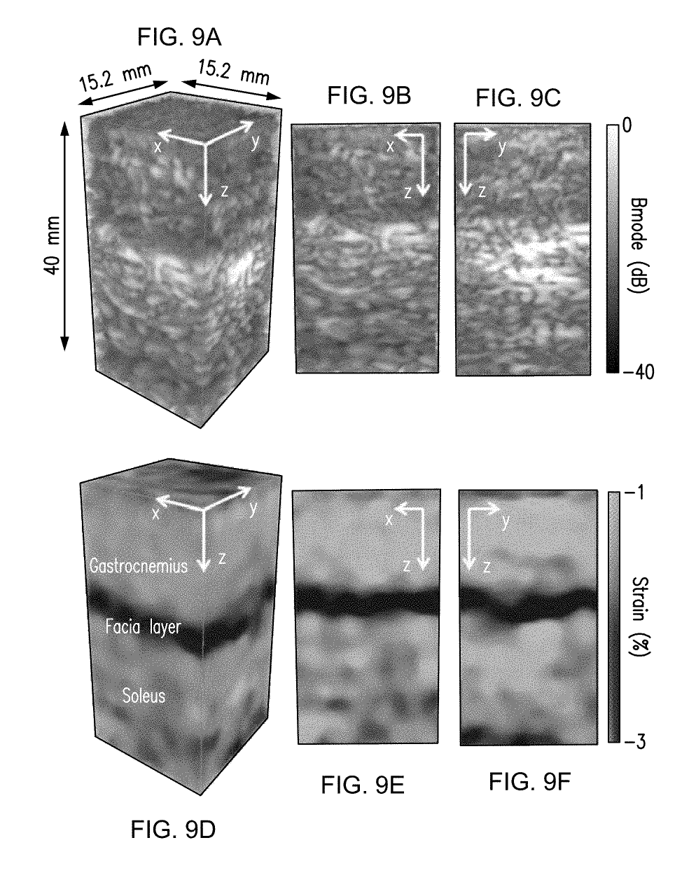

The feasability of method was then demonstrated in vivo on the upper calf of a human volunteer. From the acquisition, B-mode volumes (FIG. 9A) and slices (FIGS. 9B and 9C) were reconstructed. From the freehand compression and the transmission of 100 plane waves at 100 volumes/s, a 3D axial strain volume (FIG. 9D) were reconstructed and the softer layer (deep blue) consistent with the location of the fascia layer between the muscles of the gastrocnemius muscle and the soleus muscle (i.e., FIG. 9F) was detected. The two muscles exhibited similar strains.

Discussion

The objectives were to develop a new 3D axial strain method using plane waves at high volume rate. The feasibility of 3D quasi-static elastography based on three dimensions quasi-static elastography coupled with plane wave imaging at a high-volume rate using a 2D matrix array probe were demonstrated. Axial strains in three dimensions in phantoms, in an ex vivo biological tissues for lesion detection, and in vivo in the calf muscle of a human volunteer can be estimated.

A 3% motorized continuous quasi-static compression was combined with 3D strain imaging with plane waves, in order to acquire 100 volumes at a volume rate of 100 volumes/s. In this manner, a high-volume rate allows computation of one 3D volume of cumulative axial displacements made from 100 incremental axial displacements.

The method was validated in phantoms and showed good sensitivity. Indeed, strain differences on a two-layered phantom composed of different stiffness were detected. In addition, a 14 mm diameter stiff inclusion embedded in a soft gelatin phantom and a 14 mm diameter soft inclusion embedded in a stiff gelatin phantom were detected and visualized. The feasibility of the method in ex vivo canine liver before and after an HIFU ablation by detecting the stiffer lesion after ablation was shown. The feasibility of performing 3D quasi-static elastography with plane waves at a high-volume rate in vivo was demonstrated on the calf muscle of a human volunteer by performing 3D axial strain with a simple freehand compression.

Using only one transmit plane wave to construct an entire volume enabled high volume rates and eliminated signal decorrelation occurring when multiple acquisitions from mechanical translation of a 1D array were required to construct the volume. The acquisition time thus posed no limitation.

For in vivo applications, such as breast cancer detection, the high-volume rate used in this example will likely reduce signal decorrelations from physiologic motion such as the respiration or the heart rhythm. It also reduced artifacts from hand motions in the case of freehand scanning.

In addition, the high-volume rate enabled high temporal resolution which leaded to lower strain amplitude between consecutive volumes. In this case, the Strain Filter framework predicted an improvement in the strain estimator performance by reducing signal decorrelation and increasing the correlation coefficient. In this example a volume rate of 100 volumes/s was used but the method enables the volume rate to be increased up to 3000 volumes/s, which could be used to optimize the technique based on the Strain Filter.

In addition, the high number of volumes acquired enabled the calculation of numerous incremental axial displacement volumes in order to calculate, in this example, one volume of 3D cumulative axial displacement and one 3D cumulative axial strain volume. This is a similar approach to the technique of multicompression averaging, which has been shown to increase strain signal-to-noise ratio by decreasing random noise.

This method based on axial strain estimation could also be extended to lateral and elevational strains to correct the axial strain or to obtain the full strain tensor in biological tissues, with a high temporal resolution.

Due to the associated high-volume rates, this method could also be extended to moving organs such as the heart. Myocardial elastography, a method for the estimation of the strain distribution in the heart, could be measured in entire volumes at high temporal resolution.

The implementation on GPUs enabled us to obtain the 3D B-mode and the 3D axial strain in a few seconds following the acquisitions. This method thus can be implemented in real time.

Conclusion

3D quasi-static elastography with plane waves at high volume rate were developed and operated. By transmitting 100 plane waves at a volume rate of 100 volumes/s, the axial strain distribution in a two-layer gelatin phantom of two different stiffness, a soft inclusion embedded in a stiff gelatin phantom, and a stiff inclusion embedded in a soft gelatin phantom throughout the entire volume in three dimensions were detected. Moreover, the 3D axial strain in an ex vivo canine liver before and after HIFU ablation were estimated and the axial strain distribution in three dimensions to detect the lesion were mapped. Finally, in vivo feasibility of 3D quasi-static elastography with plane waves was demonstrated on the calf of a human volunteer with a simple freehand scanning at a high-volume rates. Due to the high-volume rate, 3D elastography with plane waves for in vivo applications could have a major impact for three-dimensional elasticity imaging.

### Example 2: Three-Dimensional Cardiac Electromechanical Activation Mapping with in Silico Validation

This Example describes one exemplary method of 3D electromechanical waves imaging (EWI) with 3D ultrasound in a single heartbeat in silico.

Methods

A. In Silico Example

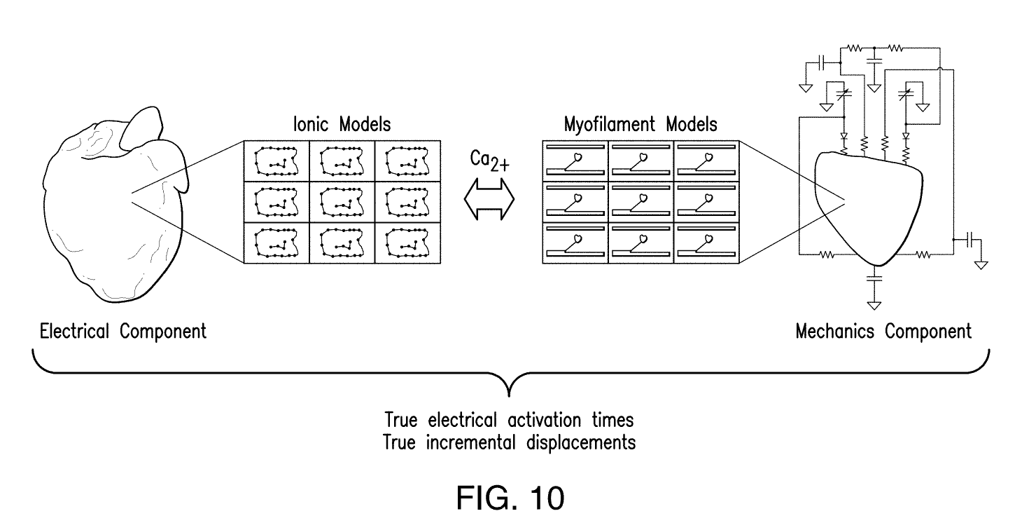

The ventricular geometry was obtained from MRI images of a healthy volunteer. Finite element simulation of an electromechanical heart model during normal sinus rhythm was performed to design electromechanical model of the human heart as shown in FIG. 10.

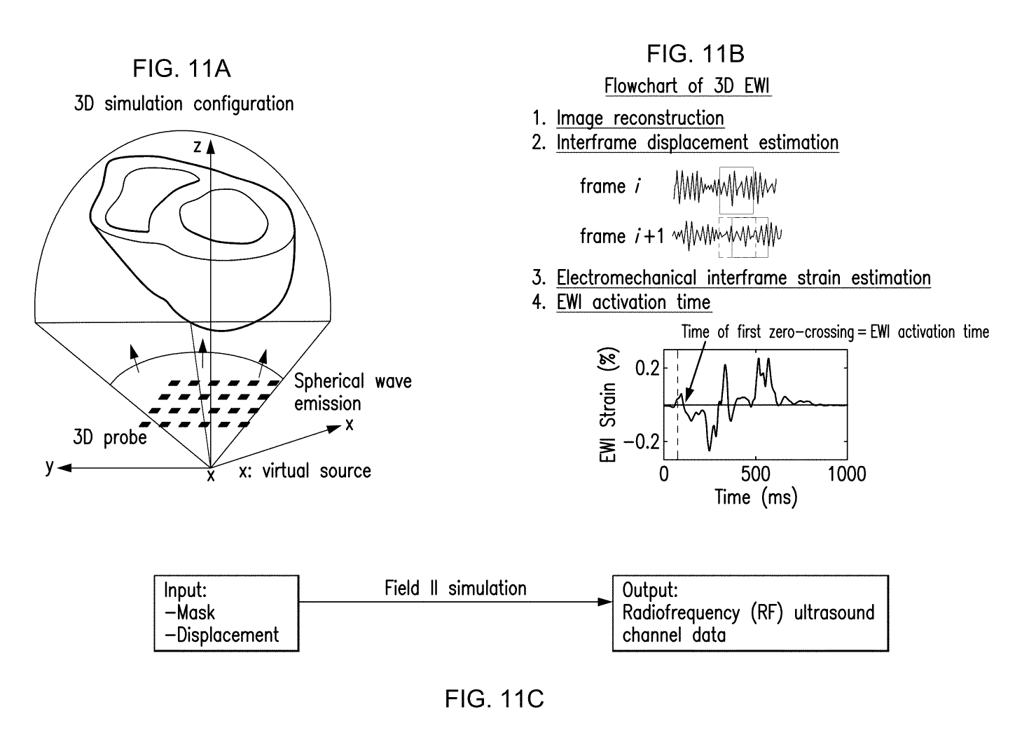

For 3D ultrasound simulation with Field II software, a 3D ultrasound probe with 32-by-32 transducer elements, a 9.6 mm² foot-print and a central frequency of 3 MHz was modeled. During the stimulation with the Field II software, a simulated numerical phantom with a ventricular mask having a 170×170×120 mm volume with 1 mm voxel size were added as inputs. Imaging sequences were set to emit 563 spherical waves at a rate of 556 volumes per second in order to reconstruct 3D volumes as shown in FIG. 11A. The radio-frequency (RF) channel data was recorded and stored in memory.

The flow chart of 3D electromechanical wave imaging (EWI) analysis is depicted in FIG. 11B. From RF data, images were reconstructed. The interframe displacements were estimated between two successive interframes to calculate electromechanical interframe strain and EWI activation time.

B. In Vivo Example

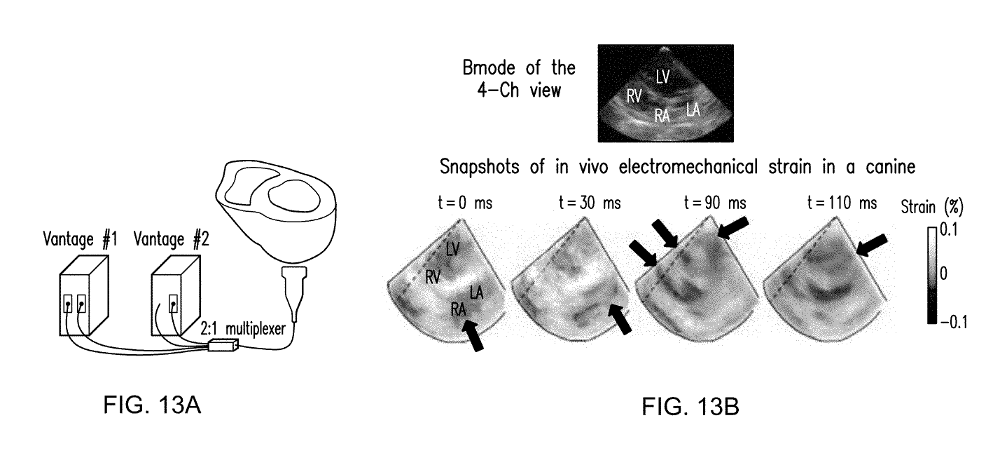

An in vivo example was also performed on the heart of an open-chest canine with normal sinus rhythm. A fully programmable ultrasound system with fully programmable channels in emission and receive was used to control a 3D ultrasound probe of 32-by-32 transducer elements. A 2:1 multiplexer was used for transmit and receive channels as shown in FIG. 13. 500 spherical waves were emitted from the 2D-matrix array probe at a rate of 500 volumes per second in order to reconstruct 3D volumes; the radio-frequency (RF) backscattered echoes from each wave were recorded and stored in memory.

Results

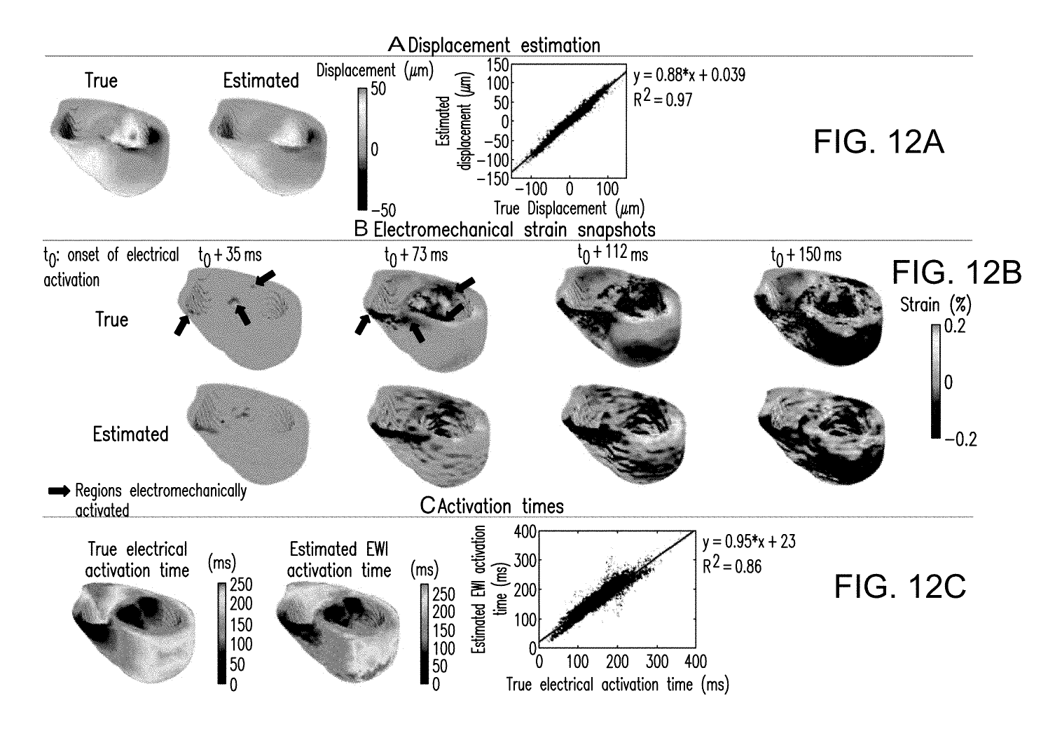

The electromechanical wave was successfully imaged with 3D ultrasound in a single heartbeat in silico. As FIG. 12 shows, the ventricular displacement and strain at a high-volume rate in a realistic human heart model were accurately estimated when compared to an electromechanical model of a human hart with true electrical activation times and incremental displacements. The estimated electromechanical and true electrical activation times were strongly correlated. For example, as shown in FIG. 12, correlation value between estimated electromechanical and true electrical activation times is R²=0.86. As shown in FIG. 13B, in vivo 3D electromechanical mapping of the heart was feasible during a single heartbeat with high frame rate and with high temporal resolution.

Discussion

As demonstrated in this Example, the disclosed systems and techniques can be used for 3D electromechanical mapping of the heart, e.g., during a single heartbeat, and can provide validation of 3D EWI in arrhythmic patients. The 3D imaging of electromechanical strain at high volume rate can be used to show an earlier contraction in the atria than in the ventricles. Moreover, the 3D electromechanical mapping can be performed in vivo to study the electromechanical strain in a heart.

### Example 3: 4D Noninvasive Cardiac Electromechanical Activation Mapping

This Example illustrates the use of the disclosed technique for noninvasive 4D mapping of the cardiac electromechanical activity in a single heartbeat. Electromechanical Wave Imaging (EWI) can noninvasively map myocardial activation of the full heart in a single heartbeat throughout all four chambers in 4D (e.g., 3D over time). This technique can image cardiac deformation resulting from electrical activation with high frame rates (for example and without limitation, 500-2000 frames per second) ultrasound to derive the electromechanical activation of the heart. At the cellular level, action potential in a cardiac cell or myocyte can be followed by cell shortening within tens of milliseconds. The propagation of myocardial shortening in the heart can be described as an electromechanical wave, as it includes mechanical deformations resulting from their local, respective electrical activations. Therefore, this technique can be referred as to electromechanical, rather than mechanical, wave imaging, in contrast with a purely mechanical wave that is generated by the mitral or aortic valve closure, for instance. Therefore, an activation map as provided by EWI can be used as a surrogate for cardiac electrical mapping, considering the strong correlation between electrical and EWI activation times. Furthermore, EWI can be beneficial at least in part because cardiac diseases manifest themselves in both electrical and mechanical aspects. EWI results from the depolarization wave and therefore has potential for cardiac activation and arrhythmia characterization. The disclosed technique can capture the activation wave throughout all four chambers in vivo.

In this example, electromechanical activation maps were presented in a normal and infarcted cardiac model in silico and in a paced canine heart in vivo. Noninvasive 4D electromechanical activation mapping in a human subject was also performed. This noninvasive 4D mapping technique can be readily applied in the clinic for direct visualization of electromechanical activation of the heart at the early onset of heart dysfunction, and thus result in timely and effective treatment thereof.

Method and Materials

Computer Heart Simulation:

The disclosed electromechanical heart model consists of an electrophysiological model and a mechanical model. The electrophysiological model represents the activation and propagation of cardiac action potentials by using a reaction-diffusion partial differential equation simulated by the software CARP (CardioSolv LLC). The mechanical model describes the heart deformation by the balance-of-force equation in which the active contraction force of myofilaments drives the cyclic heart motion; myocardial tissue was regarded as an orthotropic, hyperelastic, and nearly-incompressible material. The electrophysiological and mechanical models were coupled at the cellular level: the membrane ionic kinetics regulates the intracellular calcium transient, which dictates myofilament contraction.

The electromechanical simulation was conducted based on a MRI-image-based finite element human heart model with rule-based fiber structures, with the myocardial tissue regarded as an orthotropic, hyperelastic, and nearly-incompressible material. The simulation result describes the deformation of a human heart without and with infarct, both during sinus rhythm. The inter-volume axial (apex-to-base direction) displacements obtained from the mechanical finite-element simulation were sampled at 1.8 ms.

While clinical contrast-enhanced (late gadolinium enhanced, LGE)-MRI scans of patient hearts were used to show structural remodeling associated with myocardial infarction (MI). Post-MI patient-specific models were used electrophysiological simulations. For this example, one of the post-MI patient-specific heart models was utilized. The deformation of this post-MI patient heart during sinus rhythm was simulated. For a control simulation, the infarct region was removed and the simulations repeated.

The ultrasound simulation program Field II was used to generate the ultrasound radio-frequency channel signals with a simulated transducer and a distribution of scatterers. The scatterers were uniformly distributed at random positions in the ventricles, which geometry was obtained from the mechanical finite-element simulation at each sample. In order to perform 4D ultrasound imaging, a 2D array of 32×32 transducer elements, with an inter-element spacing (or pitch) of 0.3 mm and a center frequency of 3 MHz was simulated. Increased volume-rate imaging was achieved by using parallel beamforming with diverging wave imaging, for which the entire volume can be reconstructed from a single transmitted beam. The emission of a spherical wavefront with a transmit angle aperture of 900 was achieved by placing a virtual source 4.8 mm behind the surface of the transducer. A delay-and-sum algorithm was used to reconstruct the image with an axial sampling of 32.1 μm, a sector angle of 90° and a density of one line per degree in both lateral and elevational directions. The scatterers were displaced according to the displacement field obtained from the mechanical finite-element simulation at each sample. The ventricles were imaged at each sample (1.8 ms) during an entire heartbeat, which entails an imaging rate of 556 volumes per second.

Animal Protocol:

Three male canines (24.1±1.0 kg) were premedicated with diazepam (0.2-1 mg/kg) injected intravenously and then anesthetized with an intravenous injection of propofol (2-5 mg/kg). The canines were mechanically ventilated with a rate- and volume-regulated ventilator on a mixture of oxygen and titrated 0.5-5% isoflurane. Lidocaine (30-50 μg/(kg·min)) was injected intravenously throughout the procedure to reduce the occurrence of ventricular arrhythmia. A left lateral thoracotomy was performed with electrocautery and one rib was removed to expose the heart. In the first canine, no other procedures were performed before ultrasound data acquisition during normal sinus rhythm. In the second canine, one external bipolar electrode was sutured onto the epicardial surface of the LV in the vicinity of the apical region. The epicardial electrode was connected to a function generator (AFG3022C, Tektronix, Beaverton, Oreg.), delivering a 1-V amplitude, 2-ms pulse width at a period of 400 ms. In the third canine, the proximal left anterior descending (LAD) artery was ligated to create a myocardial infarct. The thoracotomy incision was then repaired in layers and the skin closed with surgical staples. The canine was maintained during 3 days in post-operative care during the development of the infarct. Afterwards, the chest was re-opened following the same procedure as described earlier, and ultrasound and electrical data acquisition were performed during sinus rhythm and during ventricular tachycardia (VT). In order to induce VT, lidocaine injection was stopped and pacing was performed with one electrode positioned (not sutured) on the epicardial surface near the infarct border zone. The pacing signal amplitude and pulse width were similar as described above. The pacing signal period was initially set to 400 ms. Pacing was performed for 10-15 s. After the pacing was turned off, if the canine was maintained in VT, ultrasound and electrical mapping were performed. If the canine reverted to sinus rhythm, the pacing electrode was moved to a different location and pacing was resumed. The pacing signal period was decreased by step of 20 ms if VT was not induced after trying various pacing locations.

Electroanatomical mapping of the ventricular surface was performed using a bipolar catheter (TactiCath, St. Jude Medical, Saint Paul, Minn.). The 3D position of the catheter was obtained from a navigation system (EnSite Precision, St. Jude Medical, Saint Paul, Minn.) using adhesive sensor patches placed on the canine.

Electromechanical Wave Imaging:

Ultrasound imaging was performed during normal sinus rhythm, epicardial pacing and ventricular tachycardia in the open-chest canines and transthoracically during normal sinus rhythm in a healthy human volunteer and during right ventricular and biventricular pacing in a heart failure patient. Informed consent was obtained from the participants. A custom-made 2D array of 32×32 ultrasound elements at 3 MHz center frequency and with a pitch of 0.3 mm (Vermon SA, France), identical to the one designed for the ultrasound simulation, was used for 4D ultrasound imaging. The 2D array, which has a total of 1024 transducer elements, was connected to two identical ultrasound scanners (Vantage, Verasonics, Kirkland, Wash.), each of which having 256 transmit/receive channels. In order to use all the elements of the 2D array, a 2:1 multiplexer, allowing for switching from one half of the array to the other half was used. Both diverging and conventionally focused wave imaging sequences were used to image the canine heart. The ultrasound probe was positioned to acquire apical views of the heart. For diverging wave imaging, similarly to the ultrasound simulations, a virtual source was placed 4.8 mm behind the surface of the transducer to allow for high volume-rate imaging. The first and the second halves of the 2D array aperture were used alternatively for each transmit-receive event at a pulse repetition frequency of 1000 Hz. Two consecutive transmit-receive events using each half of the aperture were used to reconstruct an entire image, yielding an imaging rate of 500 volumes per second. The ultrasound channel data were acquired during 2 s. A standard delay-and-sum beamforming, identical to the one used in the simulation, with an axial sampling of 32.1 μm, a sector angle of 90° and a density of one line per degree in both lateral and elevational directions was performed. 1-D (axial) normalized cross-correlation (5 mm window, 70% overlap) of beamformed RF signals was used to estimate inter-volumes displacements. The axial inter-volume strains were obtained using a least-squares estimator implemented with a Savitzky-Golay filter. The axial inter-volume strains were mapped to volume of 256×256×256 voxels. For in vivo imaging, segmentation was performed from focused 2D ultrasound images acquired immediately after the diverging wave imaging sequence. Focused 2D image acquisitions were performed using one quarter of the 2D array aperture (32×8) and by steering the focused beam in the lateral direction over a 90° sector angle with a beam density of one line per degree while the elevational focus was set to a depth of 70 mm. The epicardial and endocardial contours as well as the atrial and ventricular septum were manually segmented from the 2D ultrasound image. The segmentation in the elevational direction was performed by assuming central symmetry of the left atria and ventricle and of the epicardial and endocardial contours at each depth. By orienting the transducer in the apex-to-base direction, the direction of propagation of the ultrasound beam was approximately aligned with the longitudinal direction of the heart. Therefore, myocardial contraction, which is associated with longitudinal shortening, corresponded to negative axial strain. Therefore, local longitudinal shortening was characterized by negative axial strains. The onset of longitudinal shortening, resulting from electrical activation, was determined by the time of first positive-to-negative zero-crossing of the axial strain curves after a reference time point and was defined as the electromechanical activation time. A random selection of 1,000,000 voxels from the reconstructed myocardial volume was performed and for each voxel selected, the time of first positive-to-negative zero-crossing after the reference time point was automatically computed. During normal sinus rhythm, the reference time point was set to the onset of the P-wave for voxels located in the atria and to the onset of the QRS complex for voxels located in the ventricles. During ventricular pacing, the reference time point was set to the time of pacing. The electromechanical activation times were then linearly interpolated in the myocardial volume to obtain the electromechanical activation map or isochrone. The in vivo electromechanical map could be replicated for two consecutive cardiac cycles.

Results

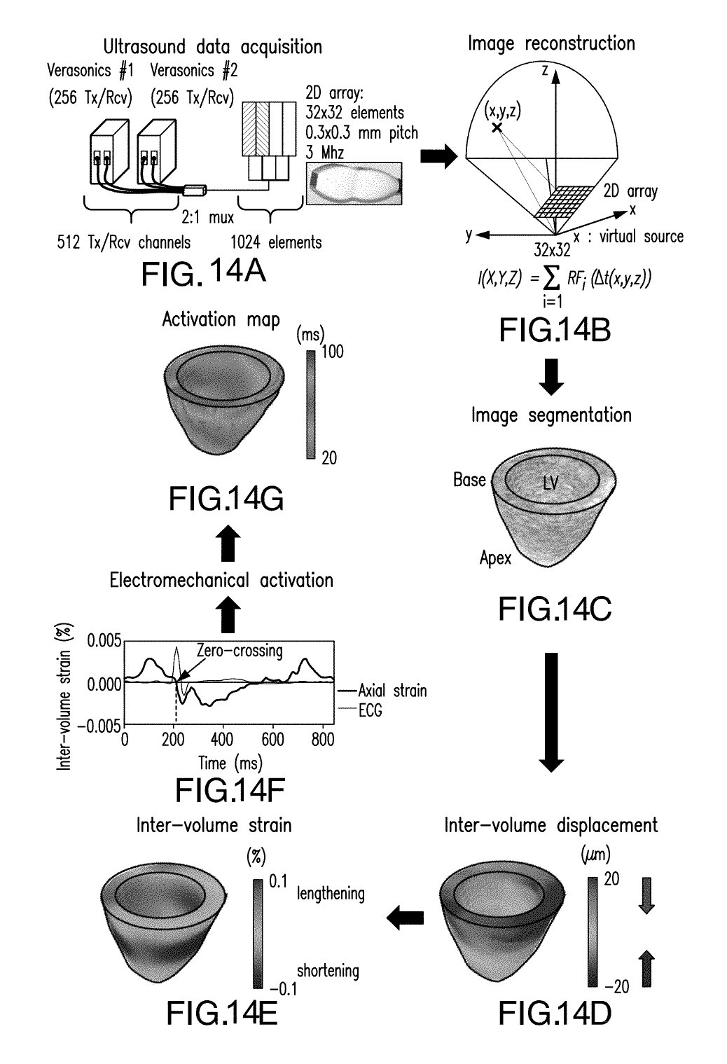

4D, increased-volume-rate EWI was capable of mapping the electromechanical activation of the entire heart noninvasively and in a single heartbeat (FIG. 14). The ultrasound data including images and radio frequency signal was acquired at an increased-frame rate, the axial displacements and the incremental strains of the heart are calculated, the zero-crossings of the strains indicating the electromechanical activation were detected for each point in order to generate an activation map. The heart was first imaged in the apical view with 4D ultrasound using emission of diverging acoustic waves to achieve increased volume-rate imaging. Inter-volume axial displacements and strains were subsequently estimated, and the local onset of contraction was defined by the time of first positive to negative zero-crossing of the temporal axial strain curve at a given location. The electromechanical activation map was then obtained by determining the local onset of contraction for each voxel of the myocardium. Validation of the electromechanical maps was performed, in silico against electrical maps and in vivo against the clinical gold-standard, i.e. electroanatomical mapping. Proof of this technique was also shown transthoracically in a normal human subject in vivo.

In Silico EWI of Human Hearts

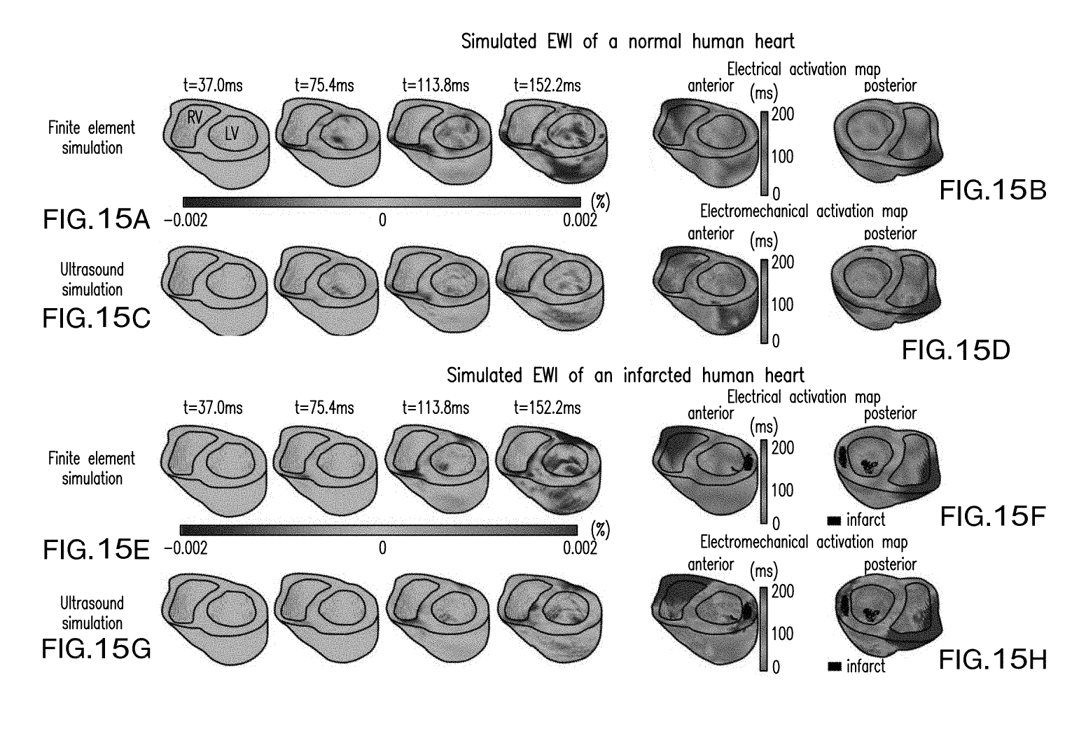

To demonstrate the feasibility of 4D EWI, an ultrasound simulation program (Field II) was used in combination with finite-element electromechanical simulation of a human heart model reconstructed from MRI images. The electrophysiological model represents the activation and propagation of cardiac action potentials by using a reaction-diffusion partial differential equation. The heartbeat was then imaged with 4D ultrasound at 556 volumes per second. Inter-volume axial displacements and strains were estimated from the ultrasound radiofrequency (RF) signals. Ventricular axial strains estimated with ultrasound were compared to their computational equivalents in the infarct-free and infarcted hearts (FIG. 15). Snapshots at different time points after the onset of electrical activation illustrate the propagation of the electromechanical wave. Negative strains, indicating shortening, correspond to myocardial contraction. In the simulated infarct-free heart, early contraction occurs in the ventricular septum, between the right and left ventricles, as well as in the anterior basal region of the right ventricle for (FIG. 15A). Myocardial contraction then propagates in both ventricles. Snapshots of the estimated axial strains (FIG. 15C) were in agreement with their true counterparts (FIG. 15A) as they exhibit a similar pattern. True electrical activation times (FIG. 15B) were also in agreement with the estimated electromechanical activation times (FIG. 15D). The electrical activation maps, or isochrones, exhibited early activation in the septal region, the endocardial layer of the anterior LV, the anterior region at the base of the RV and the posterior-septal region at the base of the LV. The electromechanical activation map obtained from EWI also points these regions as earliest sites of activation. Delayed sites of activation were found in the posterior region of the RV at the basal level, in the epicardial layer of the anterior-lateral region of the LV and in the basal region of the posterior-lateral LV. In the infarcted heart, a similar pattern of electromechanical wave propagation was identified for the benchmark (FIG. 15E) and the estimated (FIG. 15G) axial strains. Agreement was obtained between the true electrical (FIG. 15F) and the estimated electromechanical (FIG. 15H) activation times. The infarct region was not activated.

In Vivo EWI of a Canine During Sinus Rhythm

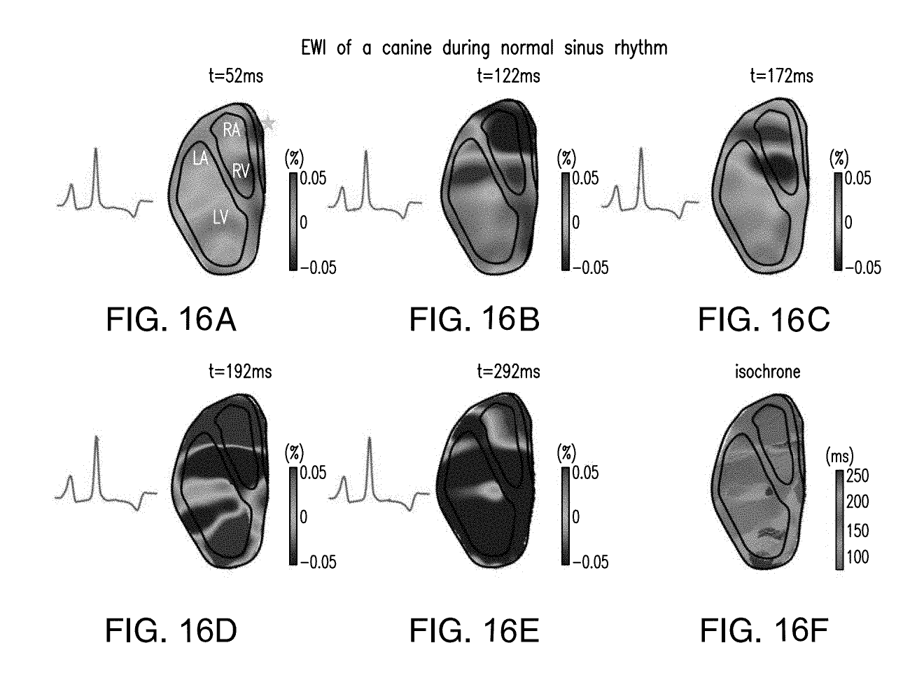

Feasibility of 4D EWI and cardiac activation mapping in a canine model were assessed. Inter-volume axial strains were imaged during a single heartbeat. Snapshots of the inter-volume axial strains at different time points of the cardiac cycle show the propagation of the electromechanical wave (FIG. 16). The earliest myocardial contraction occurs in the right atrium (FIG. 16A), during the P-wave. The electromechanical wave then propagated to the left atrium (FIG. 16B). After the onset of the QRS complex, early contraction occurred in the ventricular septum (FIG. 16C) and then propagated to both ventricles (FIGS. 16D and 16E). The propagation of the electromechanical wave was consistent with the ECG, which is a purely electrical measurement of cardiac activity. The electromechanical activation times were mapped in the canine heart (FIG. 16F). The electromechanical activation map obtained with EWI was consistent with the ECG and the known sequence of electrical activation of a normal heart.

In Vivo EWI of a Canine During Electrical Pacing

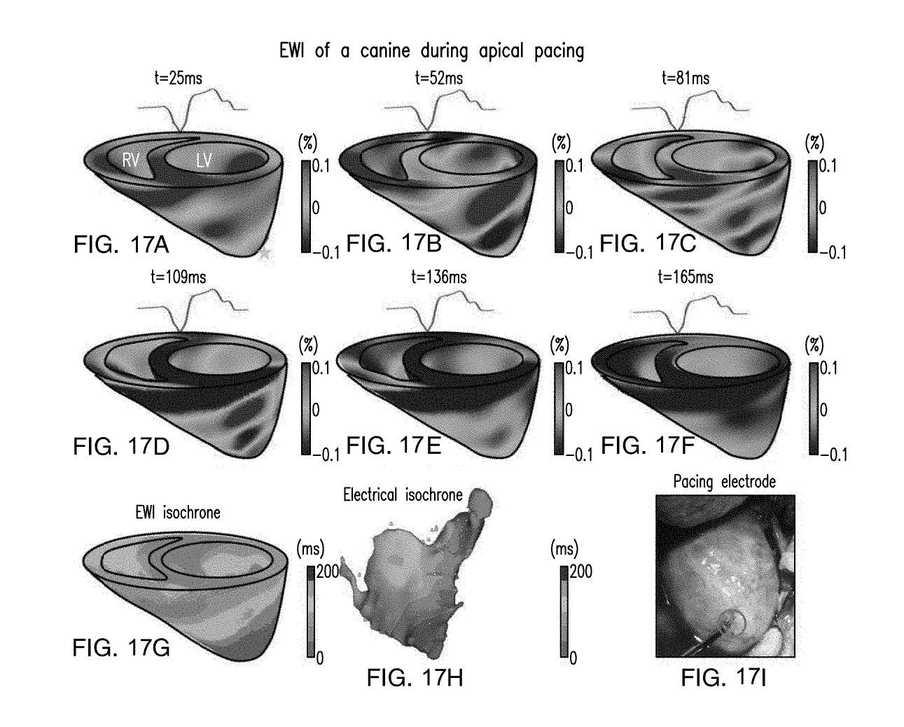

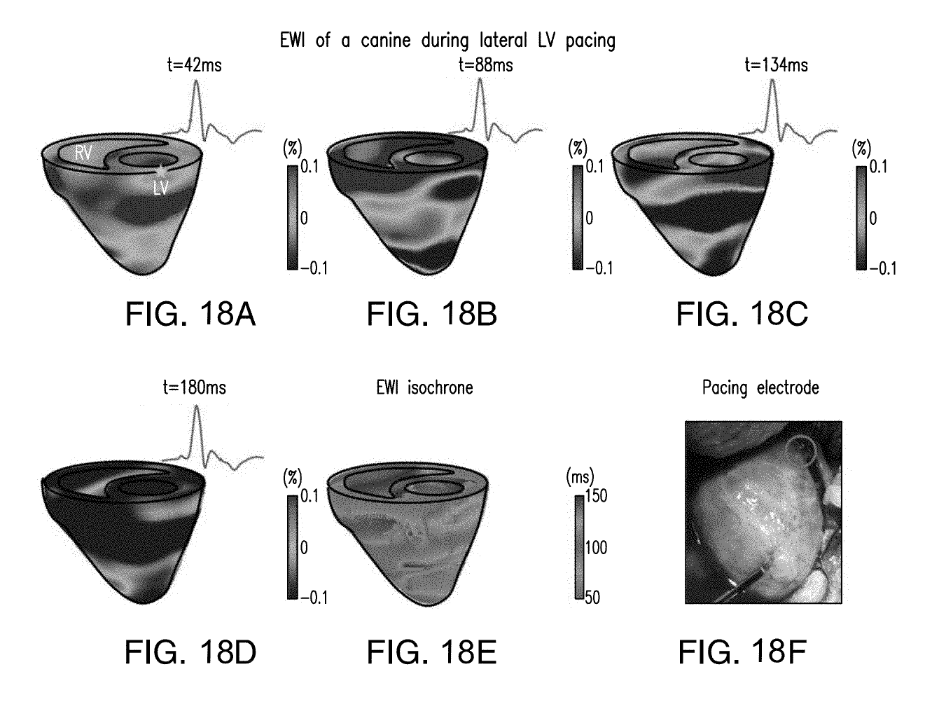

Validation of electromechanical mapping was also performed in a canine in vivo. Two pacing electrodes were sutured onto the epicardial surface of the heart: one in the anterior region at the apical level and the other one in the lateral free wall of the LV at the basal level (FIG. 17I). During apical pacing, both ventricles were imaged with increased-volume rate 4D ultrasound in a single heartbeat. After EWI data acquisition, electroanatomical mapping of the epicardial surface was performed using a single electrode mapping catheter which was positioned at various locations of the epicardium that could be reached in the open-chest canine. The earliest region of contraction was found at the apex (FIG. 17A), near the position of the sutured electrode. Then, the electromechanical wave propagated towards the base in both ventricles (FIGS. 17B, C, D, E, and F). FIG. 17F shows that 165 ms after pacing, both ventricles were fully electromechanically activated. The isochronal map obtained from EWI (FIG. 17G) was found to be in agreement with the electrical activation times obtained from electroanatomical mapping (FIG. 17H). The earliest regions of electromechanical and electrical activations are located in the apex, where the electrode was pacing from (FIG. 17I). During lateral LV free wall pacing, increased volume-rate 3D ultrasound images were acquired, and snapshots of the inter-volume axial strains showed the propagation of the electromechanical wave (FIG. 18). Two sites of early contraction were found after pacing: the lateral free wall at the basal level, where the pacing signal was delivered, and the apical region of the lateral wall. The electromechanical wave then propagated to both ventricles and the RV free fall was the last region activated. FIG. 18E shows an isochronal map, where early activation is shown in the LV free wall and late activation in the RV free wall. The position of the pacing electrode at the basal level of the LV (FIG. 18F) is in agreement with early activation in this region.

In Vivo EWI of a Normal Human Heart

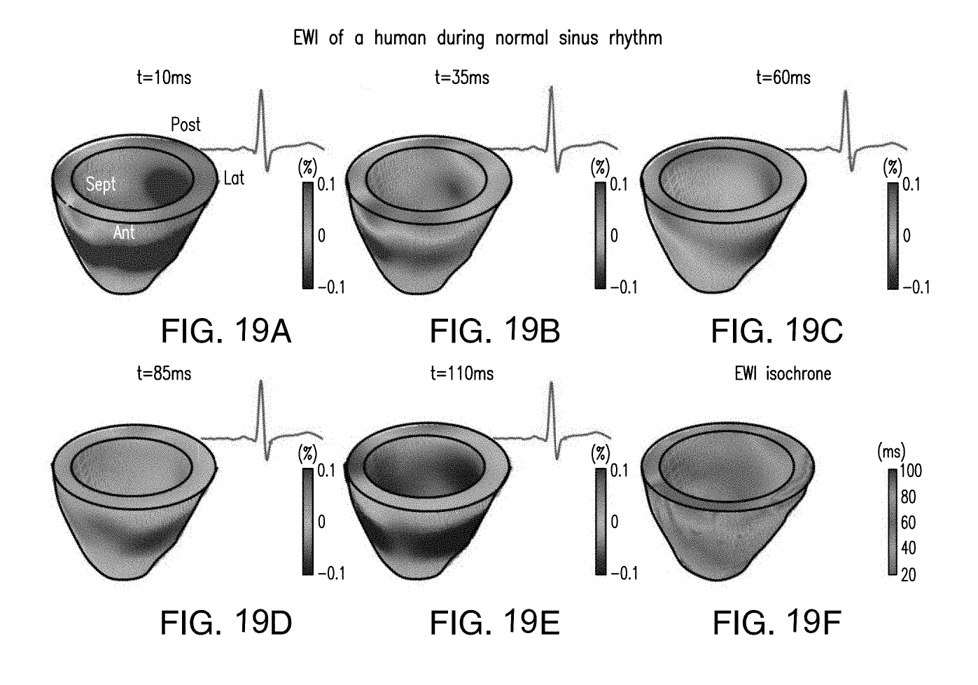

The feasibility of noninvasive 4D activation mapping of the human heart in a single heartbeat using EWI was performed in a healthy volunteer during normal sinus rhythm. Increased volume-rate ultrasound imaging was performed transthoracically and the ECG was acquired simultaneously. Snapshots of LV axial strain show early contraction in the anterior- and posterior-septal regions at the basal level after the onset of the QRS complex (FIG. 19A). Then, the electromechanical wave propagated towards the anterior and lateral region of the LV at the mid- and basal level (FIGS. 19B and 19C) and finally headed to the apex (FIGS. 19D and 19E). The electromechanical activation map (FIG. 19F) illustrates the base to apex propagation, which is consistent with what was observed in the aforementioned canine case during normal sinus rhythm and in in silico case.

In Vivo EWI of an Infarcted Canine During Sinus Rhythm and VT

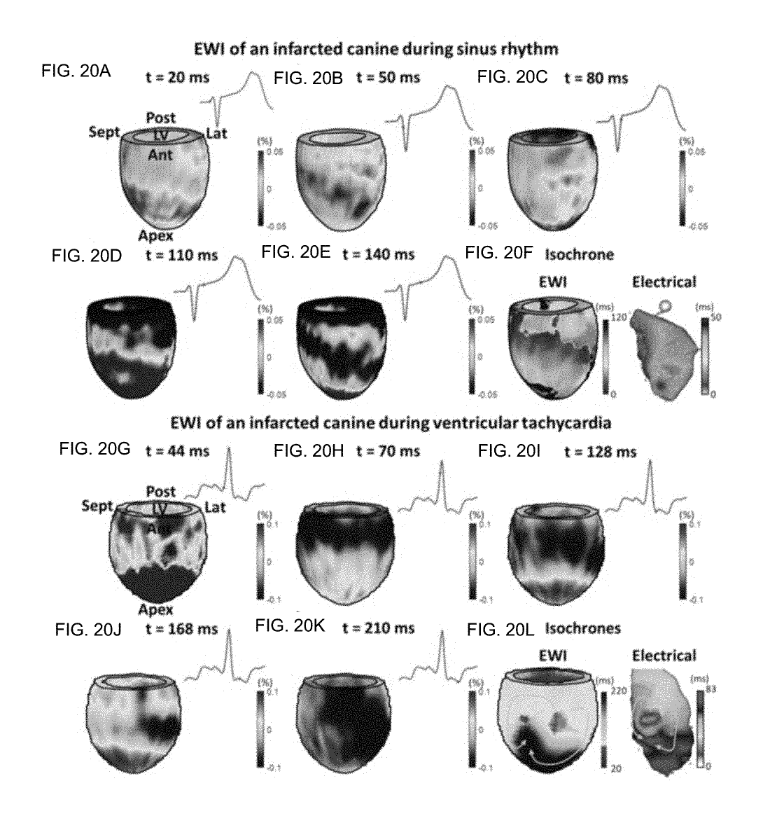

Validation of electromechanical mapping was also performed in an infarcted canine in vivo during sinus rhythm and during VT. Myocardial infarct was obtained after left anterior descending artery ligation and developed during 3 days. VT was induced after pacing the epicardial surface for 10-15 s in the infarct border zone, approximately in the anterior region at the mid-level. High-volume rate single-heartbeat 4D ultrasound and electroanatomical mapping were performed similarly as described above. During sinus rhythm, the earliest region of contraction was found at the base of the antero-spetal region (FIG. 20A) and the latest activated region was found at the apex. The propagation of the electromechanical wave was imaged (FIGS. 20A-E) and the electromechanical and electrical activation times were found to be in good agreement, showing similar early and late activated regions (FIG. 20F).

During VT, the earliest region of contraction was found at the mid-level of the anterior region (FIG. 20G) and the latest activated region was located adjacently, in the antero-septal area between the apical and mid-level. The propagation of the electromechanical wave was imaged during VT (FIG. 20G-20K) and the electromechanical and electrical activation times were also found to be in good agreement (FIG. 20L). The short distance between the late and early activated regions suggests the presence of a re-entry mechanism associated with the VT.

In Vivo EWI of a CRT Patient During RV and BiV Pacing

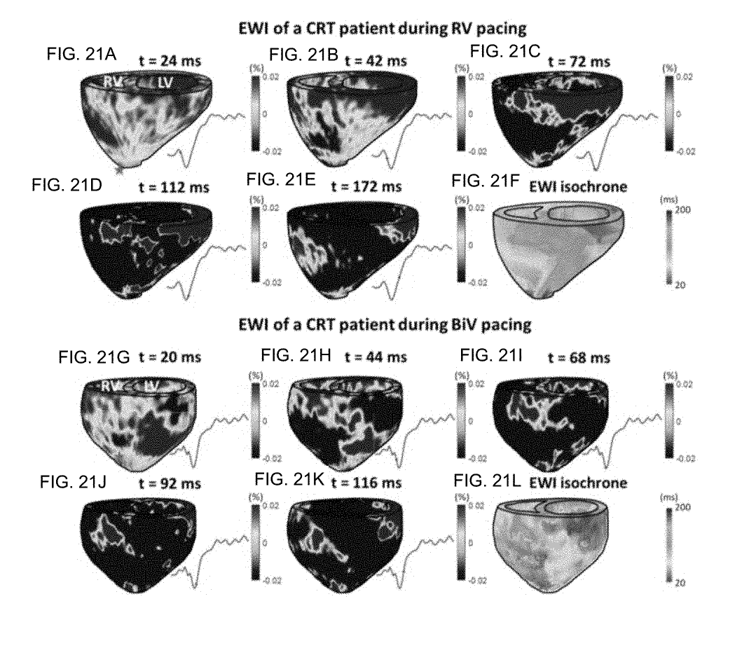

Noninvasive 4D electromechanical activation mapping of the ventricles using EWI was performed in an 84-year old male with cardiac resynchronization therapy (CRT) during RV and biventricular (BiV) pacing. The RV electrode was implanted in the apex while the LV electrode was in the coronary sinus. The electromechanical wave propagation was imaged using transthoracic high volume-rate ultrasound imaging during RV (FIG. 21A-E) and BiV (FIG. 21G-K) pacing. The EWI activation maps illustrate the activation pattern (FIG. 21F for RV pacing, FIG. 211 for BiV pacing). During RV pacing only, the earliest site of activation was found at the RV apex, which is consistent with the pacing electrode location. During BiV pacing, two sites of early activation were found: the RV apex and the antero-lateral region at the basal level. Both sites are consistent with the pacing electrodes location.

Discussion

Cardiac arrhythmia can be associated with life-threatening disease. Current methods used in the clinic to characterize cardiac arrhythmia are either not sufficiently spatially accurate, such as ECG, or invasive, superficial and time-consuming, such as electroanatomical mapping. A noninvasive 4D method to map cardiac activation of the full heart in all cardiac chambers can prove pivotal in the efficiency of cardiac arrhythmia diagnosis and treatment monitoring. 4D EWI is an imaging modality that not only can image the full heart noninvasively, but also is radiation free and cost-effective.

The electromechanical activation of the entire heart can be mapped in 4D, noninvasively and during a single cardiac cycle. The disclosed technique can be distinguished from another noninvasive method Electrocardiographic imaging which, inferring epicardial potentials from body-surface potentials, is constrained to the epicardial surface and requires wearable electrode vests as well as constructing individualized torso/heart geometries from computed tomography (CT) scans. 4D EWI can be performed with transthoracic echocardiography and therefore its implementation on a clinical ultrasound system is appropriate. It requires using diverging wave imaging as opposed to focused beams in order to image the full heart with an increased volume-rate (≥500 volumes per second). The clinical application of this technique includes cardiac ablation treatment of arrhythmia and cardiac resynchronization therapy (CRT) for heart failure. So far, locating arrhythmia is performed invasively, using single-electrode mapping catheters under fluoroscopic guidance in the clinic involving lengthy, ionizing procedures. The use of 4D EWI can decrease the procedure time as well as the risk of complications. Regarding heart failure, up to 30%-50% of patients are reported to be non-responders to CRT while CRT response strongly depends on lead placement, which is insufficiently guided. 4D EWI can be used to optimize lead placement in CRT patients by monitoring the electromechanical activation of both ventricles simultaneously. Mapping techniques presented herein are supported by computational modeling of a realistic electromechanical human heart model for both infarct-free and infarcted hearts, which can be used to assess sudden cardiac death in post-infarction patients.

The electromechanical wave corresponds to the propagation of local myocardial shortening resulting from local electrical activation. As such, it is not a direct measurement of the electrical activity of the heart. There is a delay between the electrical and the electromechanical activation in the order of tens of milliseconds, referred as to the electromechanical delay. This delay increases from early to late sites of activations. Given that it is not feasible to acquire 3D or transmural electrical activation throughout the heart, electromechanical activation maps can serve as a reliable surrogate for electrical activation times, simultaneously informing on both the mechanical and electrical activity of the heart. This technique can be employed as a routine tool for unprecedented screening as well as for diagnosis and therapy guidance for heart disease patients at the point of care.

### Example 4: 3D Myocardial Elastography

In vivo 3D strain imaging systems and techniques at high volume rates are presented. A fully programmable 2D matrix array probe to emit 2000 diverging waves at 2000 volumes/s was used to include an entire cardiac cycle. Incremental and cumulative displacement and strain were first assessed in three dimensions in the heart of open-chest canines. An external radio-frequency ablation was then performed on top of the heart on the anterior wall. Acquisitions were repeated at the ablated tissue location and incremental, cumulative displacement and strain were evaluated in three dimensions during an entire cardiac cycle on top of the lesion to highlight the tissue change.

Method and Materials

Ultrasound System

A fully programmable ultrasound system with 256 fully programmable channels in emission and receive (Vantage, Verasonics, Kirkland, USA) was used to control a 2.5 MHz ultrasonic 2D matrix array probe of 256 square elements (16×16 elements), with an inter-element spacing of 0.95 mm and a bandwith of 50% (Sonic Concepts, Bothell, USA). The maximum depth achievable with the 2D matrix array is similar to the maximum depth achievable with a regular 1D cardiac phased array probe (15-20 cm). The volume delay-and-sum beamforming and the axial strain distribution calculations were performed on a Tesla K40 GPU (Nvidia, Santa Clara, USA). 3D rendering was computed with Amira software (Visualization Sciences Group, Burlington, USA).

Setup

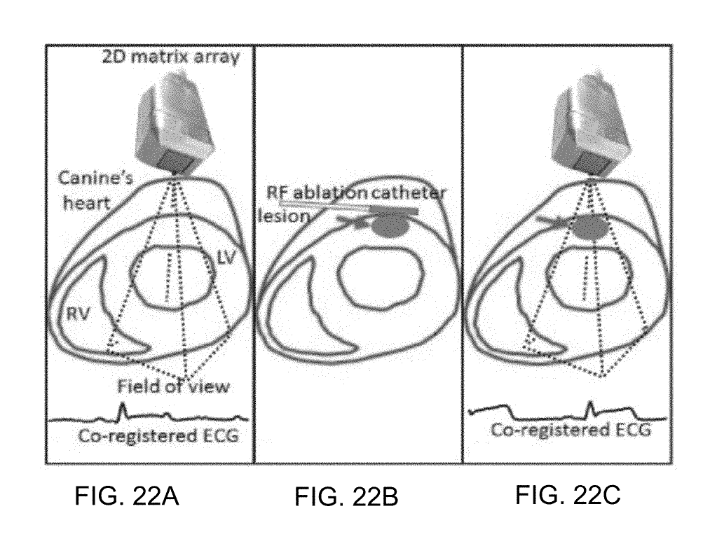

This example was conformed to the Public Health Service Policy on Humane Care and Use of Laboratory Animals and was approved by the Institutional Animal Care and Use Committee of Columbia University. Three normal male adult mongrel dogs (23-25 kg) were anesthetized with an intra-venous injection of diazepam (0.5-1.0 mg/kg) and an intra-muscular injection of hydromorphone (0.05 mg/kg). Canines were ventilated with a rate- and volume-regulated ventilator on a mixture of oxygen and titrated isoflurane (0.5%-5.0%) to maintain anesthesia. To maintain blood volume, 0.9% saline solution was administered intravenously at 5 mL/kg/h. The animal was positioned supine on a heating pad. Oxygen, peripheral blood pressure, and temperature were monitored. Standard limb leads were placed at the surface of the dogs for electrocardiogram (ECG) moni-toring and co-registration with the acquisitions (FIG. 22A). The chest was opened by lateral thoracotomy using electrocautery. A radio-frequency ablation was performed with an intracardiac catheter (St Jude Medical, Minnesota, USA) placed exter-nally on top of the anterior wall of the left ventricle of the dogs' heart (FIG. 22B). The parameters for the radio-frequency ablation system were set to 60 s and 20 W for the duration and the power respectively, to generate cylindrical lesions of approximately 1.5-2 cm diameter.

Ultrasound Acquisitions

Acquisitions were performed with the 2D matrix array positioned at a distance of 5±5 mm on top of the anterior wall of the left ventricle of the open-chest canines. The contact was ensured by ultrasonic gel. 2000 diverging waves were emitted at 2000 volumes/s before (FIG. 22A) and after (FIG. 22C) radio-frequency ablation at the lesion location. For each diverging wave, radio-frequency signals of each element were recorded at a sampling rate of 10 MHz and stored in memory. The diverging waves were generated from a virtual source positioned behind the array as at a distance of 2.6 mm to create a 40° angular aperture. All the elements were used in transmit

Image Formation and 3D Displacement and Strain Calculation

From radio-frequency acquired signals, a conventional three-dimensional delay-and-sum algorithm was used to beam-form one 3D volume from each diverging wave acquisi-tion, resulting in a total of 2000 volumes. The reconstructed depth was set to 50-70 mm with an axial sampling of 61.6 μm corresponding to a 2/10 beamforming (where 2 is the ultrasonic wavelength). The lateral distances were defined in terms of sector angles as typically done in 2D imaging of the heart. The sector angles were set at 40° and the number of reconstructed lines was set to 80 lines corresponding to a sampling of one line per 0.5° in both directions. 3D B-mode volumes were normalized and displayed using a Hilbert transform in decibels. A custom-made Matlab function was built to perform the scan conversion in 3D. This function computed remapping of a series of scan lines in spherical coordinates to a conventional ultrasound image, in Cartesian coordinates, suitable for display. Remapping was performed on a pixel grid of 256×256×256 pixels using linear interpolation via the Matlab function interp3.

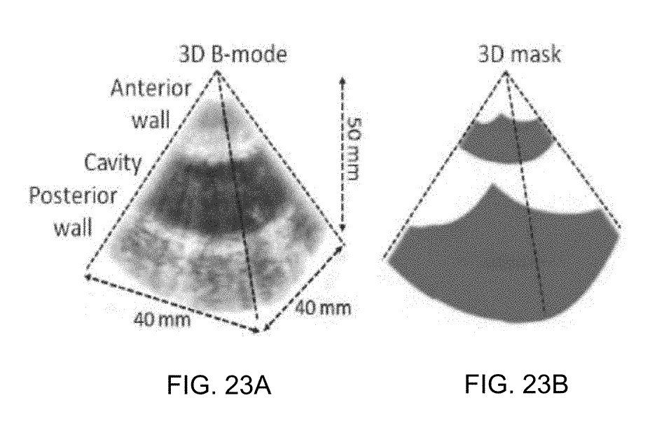

4D masks based on manual segmentation of 3D B-mode volumes over time were applied on the data to display only the tissue inside the heart wall, to remove the surrounding tissue and the signal from the cavity. An example of a 3D mask associated to a 3D B-mode is showed on FIGS. 23A and 23B. A cine-loop is provided to visualize the temporal variations of the 3D mask. The 3D axial incremental displacements between two consecutive volumes were estimated by normalized 1D cross-correlation of the RF beamformed signals with a window size of 1.2 mm (corresponding to a 22 window size and a 95% overlap). The 3D incremental axial strain was computed by applying a least-squares estimator with a kernel of 350 μm. 3D incremental axial displacement and strain were then scan-converted. A 3D median filter with a pixel kernel of 10×10×10 pixels (1.5×1.5×2.3 mm) was applied and displayed using Amira software. The central line of the displacement and strain volumes were used to display displacement and strain M-modes. To calculate the 3D cumulative Lagrangian displacement over the systolic and diastolic phases, the non-scan-converted 3D incremental axial displacement was integrated by tracking the tissue point frame-to-frame over systole and diastole respectively, according to the co-registered ECG. The 3D cumulative displacements were then scan-converted, filtered with the same 3D median filter and multiplied to the corresponding logical mask. 3D cumulative axial Lagrangian strain over the systolic and diastolic phases were computed from 3D cumulative displacement by applying the same least-squares estimator than for the incremental strain calculation. Scan conversion and 3D median filtering were applied, and the final 3D axial strain volume was displayed using Amira software. The values of cumulative displacement and strain before and after ablation on the anterior and posterior walls in three canines were averaged in a cubic region of interest positioned at the center of the walls with a size of 80×80×20 pixels (1.2×1.2×0.5 cm).

Results

Before Radio-Frequency Ablation

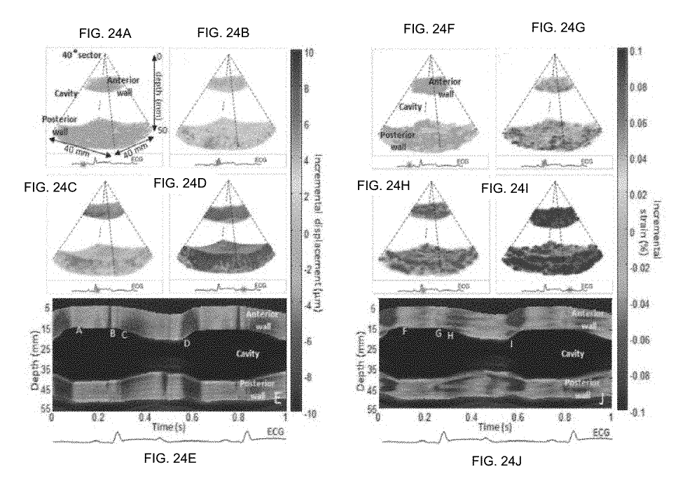

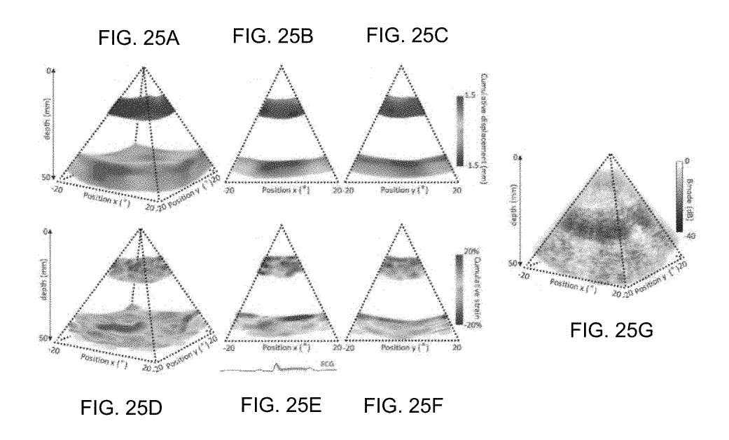

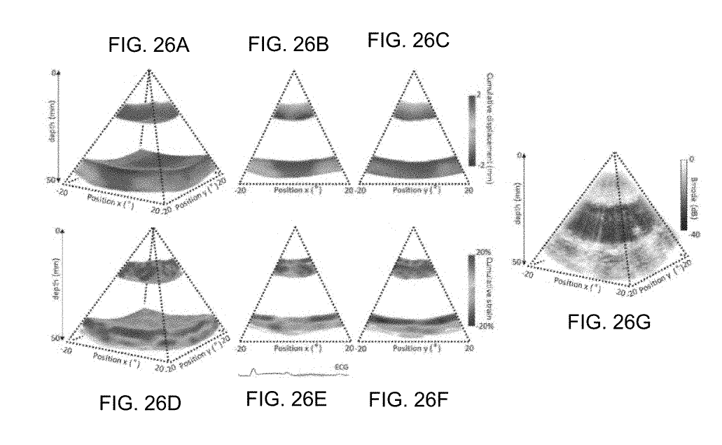

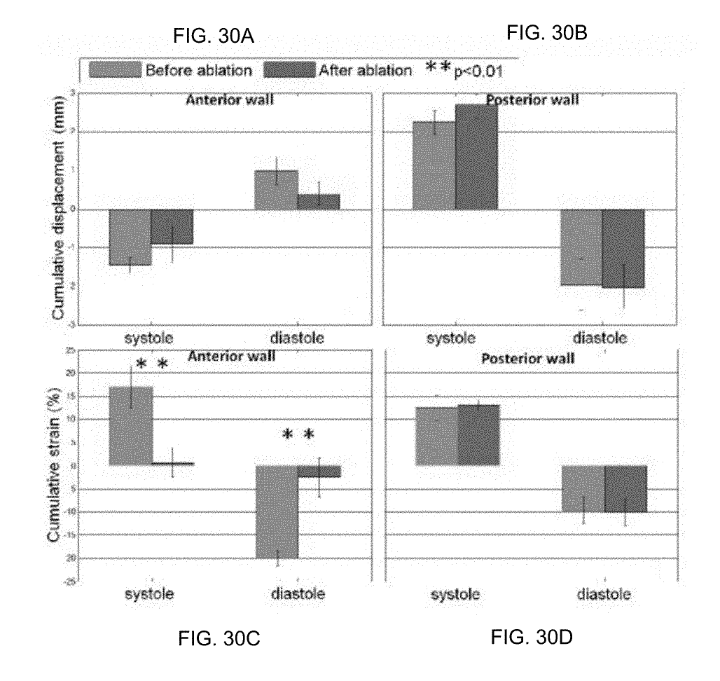

The incremental displacement and incremental strain images were first obtained before ablation in three dimensions. During mid-diastole, the heart walls (anterior and posterior walls) were found not to substantially move (FIG. 24A) and the average strain appears to be zero indicated that the heart was in the diastasis phase (FIG. 24F). During the QRS complex, fast perturbations associated with mechanical and electromechanical phenomena were observed both in the displacement and the strain (FIG. 24F and Cine-loop attached). Then, the systolic phase could be observed in the displacement as the two walls moved towards each other in order to eject the blood from the cavity (FIG. 24C). Indeed, the positive displacement indicates a motion towards the transducer whereas the negative displacement means a motion away from the transducer. The systolic phase was also defined by the contraction of the myocardium, which was observed on the incremental strains (FIG. 24H) as both walls contracted (positive strain). Finally, the diastolic phase was also highlighted. On the displacement (FIG. 24D), the two walls were found to move outward (upward for the anterior/downward for the posterior wall). On the strain, the blue color indicated relaxation of both walls (FIG. 24I). Two cine-loops associated with the 3D representation of incremental displacements and strains were attached to FIG. 24. The displacement and strain M-modes (FIGS. 24E and 24J respectively) displayed the temporal variation over the central line of the volumes and confirmed the results found. Incremental displacements were integrated during systole and diastole based on the ECG. The volume of cumulative displacement and its two associated 2D frames were displayed for systole on FIGS. 25A, 25B and 25C respectively and for diastole on FIGS. 26A, 26B and 26C. The results were consistent with the ones found with the incremental strains. The anterior wall and the posterior wall moved inwards each other during systole and outwards each other during diastole. The strain values on the anterior and posterior walls were averaged across the region of interest for the three dogs (FIG. 30). The associated cumulative strains and 2D frames were displayed on FIGS. 25D, 25E and 25F, for systole and FIGS. 26D, 26E and 26F for diastole. Similarly, the results were consistent as positive strain indicated a contraction (thickening) and negative strain indicated the relaxation (thinning).

After Radio-Frequency Ablation

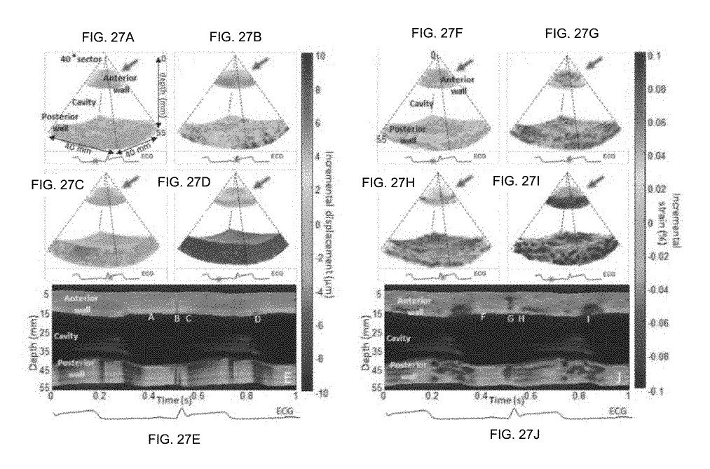

The example was then performed after a radio-frequency ablation on the anterior wall at the same location of the first acquisition. First, incremental displacements and strains were studied. The results are displayed in FIG. 27. The red arrows indicated the lesion location. During diastasis, no significant changes could be observed on the displacement (FIG. 27A) or the strain (FIG. 27F) However, during the systolic phase the anterior wall displayed a displacement (FIG. 27C) and strain (FIG. 27H) that tend to zero whereas the posterior wall displayed a normal function. The ablated tissue was thus found to become passive as it was unable to move or contract further. This result was confirmed during the diastolic phase has the anterior wall exhibited displacement and strain that also tend to zero (FIGS. 27D and 27I respectively) in opposition to the posterior wall. Two cine-loops associated to the 3D representation of incremental displacements and strains were attached to FIG. 27. The displacement and strain temporal variations were highlighted by the M-modes (FIGS. 27E and 27J) which confirmed change of the ablated tissue.

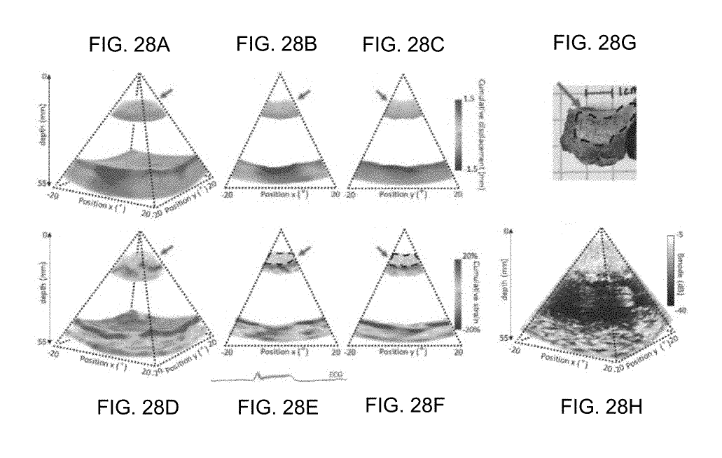

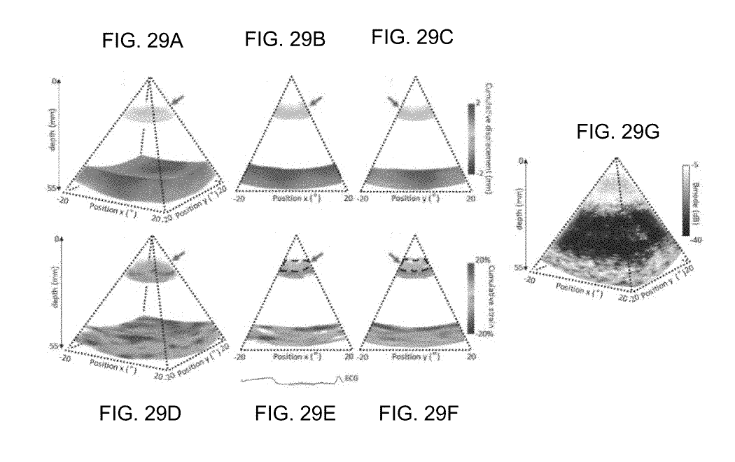

After ablation, while the posterior wall displayed the same pattern and magnitude as before the ablation. The anterior wall, however, exhibited decrease of absolute displacement at the lesion location. This decrease was depicted during systole in FIG. 28 in three (FIG. 28A) and two dimensions (FIGS. 28B and 28C) and during diastole (FIGS. 29A, 29B, and 29C). The cumulative strains were also found to be close to zero at the lesion location, during systole (FIGS. 28D, 28E, and 28F) and diastole (FIGS. 29D, 29E, and 29F) after radio-frequency ablation. Depth of the lesion on the strain map was in good quantitative agreement with gross pathology of the lesion. The results obtained for cumulative displacement and strain over diastole and systole before and after ablation for the three dogs are summarized in FIG. 30. A substantial decrease in both the absolute strain and displacement were found after ablation during systole and diastole (FIGS. 30A and 30C). However, no significant change was measured on the posterior walls (FIGS. 30B and D).

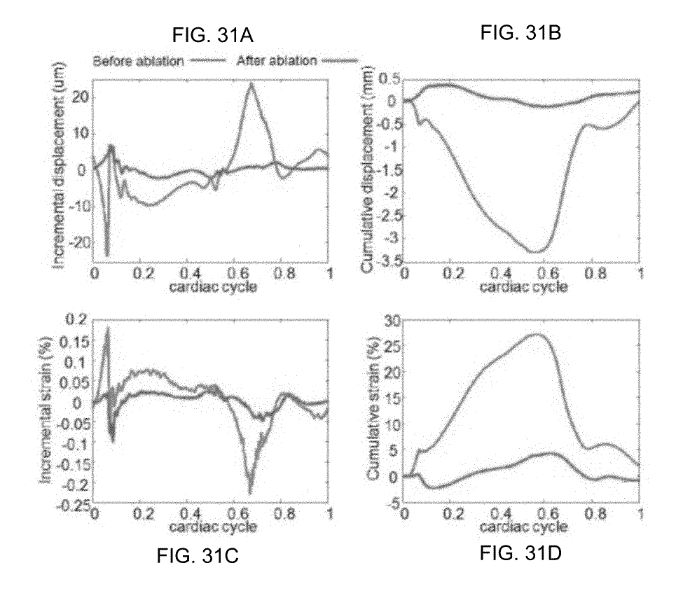

From the same data, incremental and cumulative displacement plots (FIGS. 31A and 31B) as well as incremental and cumulative strain plots (FIGS. 31C and 31D) can be assessed at each point of the volume.

Discussion

In this example, systems and techniques for imaging the heart in vivo at very high volume rates to assess tissue displacement and strain of the myocardium before and after a radio-frequency ablation lesion are described.

2000 diverging waves were emitted at 2000 volumes/s to cover an entire cardiac cycle at high volume rate. A 2D matrix array probe with 16×16 elements fully programmable placed directly on top of the heart of N=3 open-chest canines, was used for this example. With the acquired data, conventional three dimensional B-modes can be constructed for each transmit. M-modes and 3D B-modes were used to generate masks in order to display only the results corresponding to the anterior and posterior walls of the myocardium. Incremental displacements and incremental strains were successfully assessed in each voxel of the volumes using a radio-frequency cross-correlation technique. Different phases of the cardiac cycle were identified including the diastolic and systolic phases. Such a high volume rate also enabled the visualization in three dimensions of fast propagation phenomena during the QRS complex. Such phenomena associated to mechanical and electromechanical activities have been visualized in one or two dimensions in numerous studies. In this example, they were observed in three dimensions for the first time. From the incremental displacement volumes, cumulative displacement and cumulative strain volumes were computed during the diastolic and the systolic phases. On average, 20.0%/f 1.5% thinning during diastole and 17.0%/±4.6% thickening during systole were measured, which was found to be consistent with the values found in literature.

In addition, an external radio-frequency ablation was per-formed with an intracardiac catheter placed directly on top of the anterior wall of the left ventricle at the location of the first acquisition. In this example, visualization of the strain along the beam propagation direction and the strain changes in the left ventricle in vivo is shown. RF lesioning of the left ventricle is sometimes warranted in that region of the left ventricle in the case of ventricular tachycardia. In order to ensure imaging of the lesion by knowing its exact location on the anterior wall after formation, open-chest cardiac imaging needed to be performed by placing the array above the lesion. After the ablation, an identical ultrasound acquisition was performed by positioning the 2D matrix array at the lesion location. No significant differences were found in the non-ablated, posterior wall in terms of displacement or strain. However, strong differences were observed in the anterior wall at the lesion location during the cardiac cycle. The displacement and strain tend to zero as the necrotic tissue became stiffer. In this example, assessing local strain differences induced by a radio-frequency ablation in volumes is shown with a high-volume rate. Diverging wave emission thus can increase the volume rate by two orders of magnitude without trade-off on sector size or beam density. Such a high volume rate would enable to track the mechanical waves linked to the electrical activation of the heart during the QRS complex to perform electromechanical wave imaging in three dimensions. Another advantage of the technique, is the capability to assess tissue motion at high temporal resolution in each voxel of the volume. The total processing time from the RF signal acquisition to the final 3D cumulative strain volume with the sampling and depth described in the method was 83 seconds. The processing time could be decreased to a few seconds by processing only RF signals associated to the systolic (or diastolic) phase and by minimizing the axial and lateral sampling.

In this example, imaging the heart with a 2D matrix array probe at high volume rate (2000 volumes/s) using diverging emissions to assess incremental displacements and strains throughout the entire cardiac cycle in entire three-dimensional volumes in three open-chest canines was demonstrated. Rapid events propagating during the QRS complex as well as the different phases of the myocardial strain during the entire cardiac cycle were visualized. Cumulative displacements and strains were quantified before and after radio-frequency ablation in the anterior wall of the myocardium. Strong differences were measured at the lesion location highlighting the necrosis of the tissue after ablation. Thus, the 3D myocardial strain elastography method can measure the local strain distribution in three dimensions in patients and obtain ablation volumes in real-time.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein.

The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the methods and systems of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for ultrasound elastography, comprising:
emitting at least one non-focused wave to a target;
obtaining at least two Radio Frequency (RF) signals from the non-focused wave;
beamforming 3D volumes from the RFs;
calculating at least two 3D displacements by comparing each 3D volume to a reference volume; and
integrating the 3D displacements to create a 3D cumulative axial strain volume.

2. The method of claim 1, wherein the non-focused wave comprises a plane wave.

3. The method of claim 1, wherein the non-focused wave is emitted at a rate of at least 100 volumes/sec.

4. The method of claim 1, wherein the method further comprises providing a compression on the target prior to the emitting.

5. The method of claim 4, wherein the compression comprises one or more of a natural compression, a motorized compression, a manual compression, a compression by an external source, and a compression by an ultrasound probe.

6. The method of claim 1, wherein the RF is recorded at a frequency of from about 2.5 MHz to about 10 MHz.

7. The method of claim 1, wherein the beamforming further comprises performing a delay- and sum beamforming.

8. The method of claim 1, wherein the 3D displacement comprises an axial, a lateral, and/or an elevational direction.

9. The method of claim 1, wherein the 3D displacement is configured to be calculated between two successive volumes.

10. The method of claim 1, wherein the method further comprises determining a Lagrangian strain tensor.

11. The method of claim 1, wherein the method is configured to detect and treat a breast cancer.

12. The method of claim 1, wherein the method is configured to monitor a myocardial function.

13. A system for ultrasound elastography, comprising:
a 2D matrix array probe comprising an ultrasound transducer configured to emit at least one non-focused wave on a target;
a signal processor, coupled to said probe, configured to:
obtain at least two Radio Frequency (RF) signals from the non-focused wave;
beamform 3D volumes from the RF;
calculate at least two 3D displacement by comparing each 3D volume to a reference volume; and
integrate the 3D displacements to create a 3D cumulative axial strain volume.

14. The system of claim 13, wherein the 2D matrix array probe comprises a plurality of elements

15. The system of claim 14, wherein the 2D matrix array probe comprises 256 elements (16×16) or 1024 elements (32×32).

16. The system of claim 13, wherein each of the 2D matrix array is configured to emit a non-focused wave at rate of 100 waves per second.

17. The system of claim 16, wherein the non-focused wave includes a plane wave.

18. The system of claim 13, wherein the ultrasound system has a central frequency of about 2.5 MHz.

19. The system of claim 13, wherein the 2D matrix array probe is programmable.

20. The system of claim 13, wherein the 2D matrix probe is configured to receive at least two Radio Frequency (RF) signals from the non-focused wave and transmit the RF signals to the signal processor.

21. The method of claim 1, further comprising determining an electromechanical activation by beamforming the RF signals to calculate inter-volumes displacements and strains.

22. The system of claim 13, wherein the system includes a 2:1 or 4:1 multiplexer.

23. The system of claim 13, wherein the system is configured to perform a diverging imaging sequence and/or a focused wave imaging sequence.

24. The system of claim 13, wherein the signal processor is further configured to generate a heart model for an electromechanical simulation and an ultrasound simulation based on the series of image frames and the RF signals corresponding to a location in a target heart.
